(19) United States
(12) Patent Application Publication
(10) Pub. No.: US 2024/0009280 A1
(43) Pub. Date: Jan. 11, 2024

(54) RECOMBINANT ALKALINE PHOSPHATASE FOR USE IN TREATING ACUTE RESPIRATORY DISTRESS SYNDROME

(71) Applicant: AM-Pharma B.V., Utrecht (NL)

(72) Inventors: Jeroen ELASSAISS-SCHAAP, Houten (NL); Erik Jan VAN DEN BERG, Vught (NL)

(21) Appl. No.: 18/044,549

(22) PCT Filed: Sep. 9, 2021
(86) PCT No.: PCT/NL2021/050547

§ 371 Date: Mar. 8, 2023

(30) Foreign Application Priority Data
Sep. 9, 2020 (EP) 20195269.4
Jun. 10, 2021 (EP) 21178892.2

(51) Int. Cl. A61K 38/46; C12N 9/16; A61P 11/00

(52) U.S. Cl. CPC A61K 38/465; C12N 9/16; A61P 11/00; C12Y 301/03001

(57) ABSTRACT

The present disclosure relates to the use of alkaline phosphatases, and in particular improved alkaline phosphatases such as RecAP, for the prevention, treatment, cure, or amelioration of the symptoms of acute respiratory distress syndrome (ARDS). The application relates to methods of preserving lung function, shortening the duration of mechanical ventilation therapy, increasing a the P/F ratio.

The invention relates to the field of medicine. In particular, the present invention relates to the use of alkaline phosphatases, and in particular improved alkaline phosphatases such as RecAP, for the prevention, treatment, cure, or amelioration of the symptoms of acute respiratory distress syndrome (ARDS). The invention also relates to methods of preserving lung function, shortening the duration of mechanical ventilation therapy, increasing the P/F ratio.

### BACKGROUND

Acute Respiratory Distress Syndrome (ARDS) is a disfunction of the respiratory function of the lung, characterized by widespread inflammation of the lungs. Symptoms include shortness of breath, rapid breathing and bluish skin coloration (Fan, E; Brodie, D; Slutsky, A S (20 Feb. 2018). “Acute Respiratory Distress Syndrome: Advances in Diagnosis and Treatment”. JAMA. 319 (7): 698-710). The overall prognosis of ARDS is poor, with mortality rates of approximately 40% (Lewis, Sharon R.; Pritchard, Michael W.; Thomas, Cannel M.; Smith, Andrew F. (Jul. 23, 2019). “Pharmacological agents for adults with acute respiratory distress syndrome”. The Cochrane Database of Systematic Reviews. 7: CD004477). For those who survive ARDS, a decreased quality of life is common (Matthay, MA; Zemans, R L; Zimmerman, GA; Arabi, YM; Beitler, JR; Mercat, A; Herridge, M; Randolph, AG; Calfee, CS (14 Mar. 2019). “Acute respiratory distress syndrome”. Nature Reviews. Disease Primers. 5 (1): 18).

Globally, ARDS affects more than 3 million people a year (Fan E et al (2018)). Although the terminology of “adult respiratory distress syndrome” has at times been used to differentiate ARDS from “infant respiratory distress syndrome” in newborns, the international consensus is that “acute respiratory distress syndrome” is the best term because ARDS can affect people of all ages (Bernard G, Artigas A, Brigham K, Carlet J, Falke K, Hudson L, Lamy M, Legall J, Morris A, Spragg R (1994). “The American-European Consensus Conference on ARDS. Definitions, mechanisms, relevant outcomes, and clinical trial coordination”. Am J Respir Crit Care Med. 149 (3 Pt 1): 818-24). There are separate diagnostic criteria for children and for those in areas of the world with fewer resources (Matthay M A et al (2019)).

The annual rate of ARDS is generally 13-23 people per 100,000 in the general population. ARDS is more common in people who are mechanically ventilated with acute lung injury (Lewandowski K, Lewandowski M (2006). “Epidemiology of ARDS”. Minerva Anestesiol. 72 (6): 473-7). ARDS rates increased in 2020 due to COVID-19 (Guo, YR; Cao, QD; Hong, ZS; Tan, YY; Chen, SD; Jin, HI; Tan, KS; Wang, DY; Yan, Y (13 Mar. 2020). “The origin, transmission and clinical therapies on coronavirus disease 2019 (COVID-19) outbreak—an update on the status”. Military Medical Research. 7 (1): 11; Solaimanzadeh, I (20 Mar. 2020). “Acetazolamide, Nifedipine and Phosphodiesterase Inhibitors: Rationale for Their Utilization as Adjunctive Countermeasures in the Treatment of Coronavirus Disease 2019 (COVID-19)”. Cureus. 12 (3): e7343).

Worldwide, and in the absence of a viral pandemic such as COVID-19, severe sepsis is the most common trigger causing ARDS (Goldman, Lee (2011). Goldman's Cecil Medicine (24th ed.). Philadelphia: Elsevier Saunders. p. 635. ISBN 978-1437727883). Other triggers include mechanical ventilation, pneumonia, Gilchrist's disease, drowning, circulatory shock, aspiration, trauma—especially pulmonary contusion—major surgery, massive blood transfusions (Laar, Alexander P. J.; Binnekade, Jan M.; Prins, David; van Stein, Danielle; Hofstra, Jorrit J.; Schultz, Marcus J.; Juffermans, Nicole P. (March 2010). “Risk factors and outcome of transfusion-related acute lung injury in the critically ill: A nested case-control study*”. Critical Care Medicine. 38 (3): 771-778), smoke inhalation, drug reaction or overdose, fat emboli and reperfusion pulmonary edema after lung transplantation or pulmonary embolectomy.

Next to the treatment of the underlying cause, such as antibiotics, or antiviral or anti-inflammatory drug, ARDS is usually treated with mechanical ventilation in the intensive care unit (ICU). Mechanical ventilation is usually delivered through a rigid tube which enters the oral cavity and is secured in the airway (endotracheal intubation), or by tracheostomy when prolonged ventilation (≥2 weeks) is necessary. The role of non-invasive ventilation is limited to the very early period of the disease or to prevent worsening of respiratory distress in individuals with atypical pneumonias, lung bruising, or major surgery patients, who are at risk of developing ARDS. No particular ventilator mode is known to improve mortality in ARDS, which is between 35-50% (Fan E et al (2018)). There is thus a need for methods of treatment of ARDS.

### BRIEF SUMMARY

The present disclosure provides methods to treat acute respiratory distress syndrome (ARDS) in a subject in need thereof comprising administering an effective amount of alkaline phosphatase (AP) to said subject. In a preferred embodiment, the AP is administered in at least one 500 U/kg to 2,000 U/kg dose.

The severity of ARDS is typically categorized in “mild”, “moderate”, and “severe” ARDS. Mild, moderate, and severe ARDS is herein defined by the following criteria:

decreased PaO₂/FiO₂ ratio (PaO₂/FiO₂ ratio is the ratio of arterial oxygen partial pressure (PaO₂ in mmHg) to fractional inspired oxygen (FiO₂ expressed as a fraction, not a percentage) and is further referred to as P/F ratio) of:
>200 and ≤300 mmHg (>26.66 kPa and ≤40.00 kPa): mild ARDS;
>100 and ≤200 mmHg (>13.33 and ≤26.66 kPa): moderate ARDS;
≤100 mmHg (≤13.33 kPa): severe ARDS;
   wherein the P/F ratio is measured using a minimum positive end expiratory pressure (PEEP) of 5 cmH₂O.

In some aspects, the subject has severe or moderate ARDS according to the above definition (i.e. P/F ratio ≤200 mmHg using minimum PEEP of 5 cmH₂O) prior to treatment with AP. In some aspects, the subject has severe ARDS (i.e. P/F ratio ≤100 mmHg using minimum PEEP of 5 cmH₂O) prior to treatment with AP. In some aspects, the subject has moderate ARDS (i.e. P/F ratio 100-200 mmHg using minimum PEEP of 5 cmH₂O) prior to treatment with AP. In some aspects, ARDS is associated with and/or due to sepsis. In other aspects, the ARDS is associated with and/or due to a viral infection, preferably an infection with a coronavirus, more preferably an infection with a severe acute respiratory syndrome (SARS)-related coronavirus. In some aspects, the AP is a human AP. In some aspects, the AP is a recombinant AP. In some aspects, the recombinant AP is chimeric. In some aspects, the chimeric AP has at least 70%, at least 75%, at least 80%, at least 85%, at least 90%, at least 95%, at least 96%, at least 97%, at least 98%, at least 99% or 100% sequence identity to RecAP (SEQ ID NO: 1).

In some aspects, the increase in respiratory function comprises an increase in P/F ratio of the subject with respect to the P/F ratio of the subject in the absence of treatment. In some aspects, sepsis of the subject is detected less than 96 hours prior to AP administration. In some aspects, the sepsis is detected less than 72 hours prior to ARDS detection.

In some aspects, treatment according to the invention is initiated within 24 hours after sepsis of the subject is detected. In some aspects, treatment according to the invention is initiated within 24 hours after ARDS of the subject is detected. In some aspects, AP is administered once daily. In some aspects, AP is administered intravenously. In some aspects, AP is administered in three daily doses. In some aspects, the AP dose is 0.8 mg/kg or 1.6 mg/kg of RecAP, e.g., the clinical grade RecAP used in the present disclosure. In some aspects, the AP dose is 500 U/kg or 1000 U/kg of RecAP, e.g., the clinical grade RecAP used in the present disclosure.

In some aspects, the administration of at least one dose of AP results in a shortening of duration or cessation of mechanical ventilation therapy in a subject undergoing mechanical ventilation therapy. In some aspects, the administration of at least one dose of AP results in the preservation or increase of the P/F ratio in the subject.

### BRIEF DESCRIPTION OF THE DRAWINGS/FIGURES

FIG. 1 is a table showing proposed RecAP dose groups.

FIG. 2 shows a RecAP amino acid sequence (SEQ ID NO: 1).

FIG. 3 shows a timeline of the STOP-AKI study, including dosage regimen and timeframes to determine AUC₁₋₇, RRT incidence, and mortality and MAKE.

FIG. 4 is a flowchart showing the enrollment, randomization, and follow-up of the patients in the trial. Abbreviations: AKI (acute kidney injury), AUC (time-corrected area under the curve), ECC (endogenous creatinine clearance), ICF (informed consent form), ITT (intention-to-treat), IV (intravenous), MAKE (major adverse kidney events), NS (not specified), RecAP (human recombinant alkaline phosphatase having the sequence of SEQ ID NO: 1), RRT (renal-replacement therapy), SA-AKI (sepsis-associated acute kidney injury), SCr (serum creatinine).

FIG. 5 is a table presenting the demographic and baseline characteristics of all groups in the trial, showing that there were no significant differences between groups in any baseline characteristics. For variables with missing data, summary data are based on the adjusted number. Body-mass index (BMI) is the weight in kilograms divided by the square of the height in meters. Scores on the Acute Physiology and Chronic Health Evaluation II (APACHE II) range from 0-71, with higher scores indicating greater disease severity. Scores on the Sequential Organ Failure Assessment (SOFA) range from 0-24, with higher scores indicating more severe dysfunction. Scores on the Simplified Acute Physiology Score (SAPS) range from 0-163 with higher scores indicating greater disease severity. Estimated glomerular filtration rate (eGFR) was calculated according to the CKD-EPI (Chronic Kidney Disease Epidemiology Collaboration) equation: 141*min(Scr/κ,1)α*max(Scr/κ,1)-1.209*0.993Age*1.018[if female]*1.159 [if black]. Scr is serum creatinine (mg/dL), κ is 0.7 for females and 0.9 for males, α is −0.329 for females and −0.411 for males, min indicates the minimum of Scr/κ or 1, and max indicates the maximum of Scr/κ or 1. (www.kidney.org/content/ckd-epi-creatinine-equation-2009). Acute Kidney Injury (AKI) stage was stratified according to the AKI-Network definition (www.akinet.org/akinstudies.php). Median and interquartile values, or number and percentages are depicted.

FIG. 6 is an AUC vs. dose graph showing a linear increase in RecAP concentrations. Exposure in patients in the STOP-AKI clinical trial is slightly higher compared to healthy subjects. Dose linearity and proportionality were observed over the dose range (0.4-1.6 mg/kg).

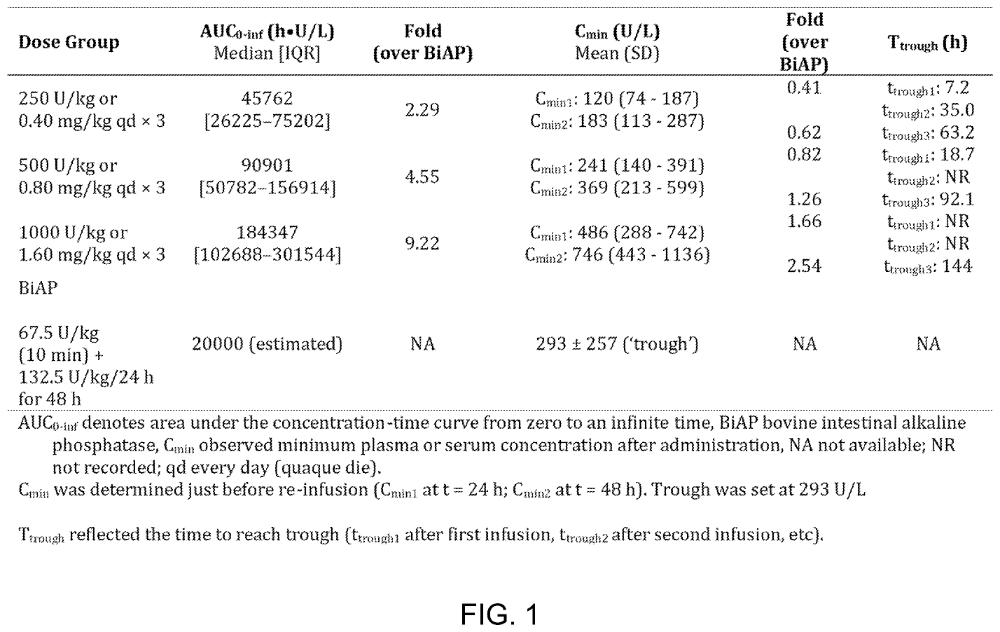

FIG. 7 is a table showing primary and secondary end points (placebo and RecAP 0.6 mg/kg group). AUC₁₋₇ denotes area under the ECC curve from day 1 through day 7, divided by 7 to provide a time-corrected clearance in ml/min. Abbreviations: CI (confidence interval), CKD-EPI (Chronic Kidney Disease Epidemiology Collaboration), ECC (endogenous creatinine clearance), eGFR (estimated glomerular filtration rate), ICU (intensive care unit), IQR (interquartile range), MAKE (major adverse kidney events), RRT (renal-replacement therapy), SOFA (Sequential Organ Failure Assessment).
§ RRT incidence indicates the percentage of patients that needed RRT following randomization.
∥ With last observation carried forward.
* MAKE 28: received RRT before or on day 28 or died before or on day 28. Proportion that met at least one of the criteria.
† MAKE 60: eGFR <60 ml/min at day 60 estimated by the CKD-EPI formula based on serum creatinine or required chronic RRT, before or on day 60 or died before or on day 60. Proportion that met at least one of the criteria.
‡ MAKE 90: 60: eGFR <60 ml/min at day 90 estimated by the CKD-EPI formula based on serum creatinine or required chronic RRT, before or on day 90 or hospitalization for new episode of AKI before or on day 90 or died before or on day 90. Proportion that met at least one of the criteria.
§§ Denotes the absolute difference (for continuous variables).
†† Denotes the odds ratio (for categorical variables).
‡‡ Denotes the hazard ratio (for events).

FIG. 8 shows primary and secondary endpoints for all treatment groups (RecAP at 0.4. mg/kg, 0.8 mg/kg, or 1.6 mg/kg doses, or placebo).

FIG. 9A shows quality of life (QoL) assessments measured by EuroQoL (EQ5D) score at ICU (left drawing) and Day 90 (right drawing).

FIG. 9B shows lung function parameters (P/F ratio, tidal volume and PEEP), depicted as medians, for the 1.6 mg/kg RecAP dose and placebo groups. Numeric values are shown below the graphics.

FIG. 9C shows liver function parameters (alanine aminotransferase, and aspartate aminotransferase activity), depicted as medians, for the 1.6 mg/kg RecAP dose and placebo groups. Numeric values are shown below the graphics.

FIG. 10 shows the results of a post hoc multivariate analysis.

FIG. 11 shows treatment-emergent adverse events observed during the STOP-AKI study.

FIG. 12 shows P/F ratio values in STOP-AKI in the population of subjects with measurable screening or Day-1 values, who were randomized into either placebo or high-dose recAP up to and including day 29. Numbers indicate the number of patients in that particular timepoint per treatment arm. The mean value is displayed together with the bootstrapped 95-% confidence intervals (CL). Time was shifted slightly per treatment group to increase the discrimination of CLs.

FIG. 13 shows fold-change of P/F ratio values in STOP-AKI in the population of subjects with measurable screening or Day-1 values, who were randomized into either placebo or high-dose recAP up to and including day 29. Numbers indicate the number of patients in that particular timepoint per treatment arm. The mean value is displayed together with the bootstrapped 95-% confidence interval. Time was shifted slightly per treatment group to increase the discrimination of CLs.

FIG. 14 shows fold-change of P/F ratio values in STOP-AKI in the population of subjects with measurable screening or Day-1 values, who were randomized into either placebo or high-dose recAP up to and including day 29. Numbers indicate the number of patients in that particular timepoint per treatment arm. The mean value is displayed together with the bootstrapped 95-% confidence interval. Time was shifted slightly per treatment group to increase the discrimination of CLs. Data of MeanBase values as per grouped ARDS criteria are displayed in different panels.

FIG. 15 shows survival of patients in STOP-AKI who were randomized into either placebo or high-dose recAP up to and including day 29. Percentage of patients that were alive, relative to day 0, are depicted for placebo or high dose AP group of patients with severe to moderate ARDS at baseline. Number of patients at day 0 was 40 in the placebo group and 42 in the high dose recap group.

### DETAILED DESCRIPTION

A goal of the present disclosure is to provide methods for preserving or improving respiratory function in a subject, especially in cases where the subject suffers from or is at risk of acute respiratory syndrome (ARDS). Another goal of the present disclosure is to prevent or shorten the duration of mechanical ventilation therapy, to preserve or increase the P/F ratio in the subject, and/or to increase survival.

Accordingly, the present disclosure relates to the use of an alkaline phosphatase (AP), e.g., RecAP, to preserve or improve respiratory function in a subject with ARDS, or in a subject who is at risk of ARDS. The disclosure provides, for example, methods for treating a subject with severe or moderate ARDS, comprising administering an AP, e.g., RecAP, to the subject. In some aspects, each dose of AP administered to the subject comprises at least 500 U/kg (0.8 mg/kg in the case of the clinical grade RecAP used). In some particular aspects, each AP dose is at least 1,000 U/g (1.6 mg/kg in the case of the clinical grade RecAP used). In some aspects the ARDS is sepsis-associated ARDS. In other aspects, the ARDS is caused or exacerbated by a viral infection, preferably by a coronaviral or orthomyxoviral infection, more preferably by a severe acute respiratory syndrome (SARS)-related coronavirus infection, such as for instance severe acute respiratory syndrome coronavirus 2 (SARS-CoV-2), the strain of coronavirus that causes coronavirus disease 2019 (COVID-19), the respiratory illness responsible for the COVID-19 pandemic in 2020. In case of an orthomyxoviral infection, the infection is preferably caused by an influenza virus, such as type A-, type B-, type C, or type D-influenza, more preferably type A- or type B-influenza.

RecAP is a chimeric AP that combines the properties of two human isoenzymes, intestinal and placental AP (Kiffer-Moreira et al. PLoS One 2014; 9:e89374). Replacing the crown domain of intestinal AP (the most biologically active isoenzyme of the two) with the crown domain of placental AP (which has the longest half-life) creates a highly stable, biologically active enzyme (Kiffer-Moreira et al. PLoS One 2014; 9:e89374). See, e.g., U.S. Patent Appl. Publ. Nos. US20170009216 and US20160250299, the disclosures of which are incorporated herein by reference in their entireties.

In some aspects, the AP, e.g., RecAP, is administered to the subject if ARDS is present and the ARDS has been determined to be moderate or severe. Thus, in some aspects of the present disclosure, the methods disclosed herein comprise measuring severity of ARDS, e.g., by determining P/F ratio, prior to treatment with AP to determine whether the subject suffers from moderate ARDS or severe ARDS.

Diagnostic criteria for ARDS have changed over time as understanding of the pathophysiology has evolved. The international consensus criteria for ARDS were most recently updated in 2012 and are known as the “2012 Berlin definition” (Ranieri V M, Rubenfeld G D, Thompson B T, Ferguson N D, Caldwell E, Fan E, Camporota L, Slutsky A S (June 2012). “Acute respiratory distress syndrome: the Berlin Definition. ARDS Definition Task Force”. JAMA. 307 (23): 2526-33; Ferguson N D, Fan E, Camporota L, Antonelli M, Anzueto A, Beale R, Brochard L, Brower R, Esteban A, et al. (October 2012). “The Berlin definition of ARDS: an expanded rationale, justification, and supplementary material”. Intensive Care Med. 38 (10): 1573-82). In addition to generally broadening the diagnostic thresholds, other notable changes from the prior 1994 consensus criteria (Bernard G, Artigas A, Brigham K, Carlet J, Falke K, Hudson L, Lamy M, Legall J, Morris A, Spragg R (1994). “The American-European Consensus Conference on ARDS. Definitions, mechanisms, relevant outcomes, and clinical trial coordination”. Am J Respir Crit Care Med. 149 (3 Pt 1): 818-24) include discouraging the term “acute lung injury,” and defining grades of ARDS severity according to the degree of decrease in the oxygen content of the blood.

According to the 2012 Berlin definition, adult ARDS is characterized by the following:
lung injury of acute onset, within 1 week of an apparent clinical insult and with progression of respiratory symptoms
bilateral opacities on chest imaging (chest radiograph or CT) not explained by other lung pathology (e.g. effusion, lobar/lung collapse, or nodules)
respiratory failure not explained by heart failure or volume overload
decreased PaO₂/FiO₂ ratio (a decreased PaO₂/FiO₂ ratio indicates reduced arterial oxygenation from the available inhaled gas):
   mild ARDS: >200 and ≤300 mmHg (>26.66 kPa and ≤40.00 kPa);
   moderate ARDS: >100 and ≤200 mmHg (>13.33 and ≤26.66 kPa);
   severe ARDS: ≤100 mmHg (≤13.33 kPa);
      The Berlin definition requires a minimum positive end expiratory pressure (PEEP) of 5 cmH₂O for consideration of the PaO₂/FiO₂ ratio. This degree of PEEP may be delivered noninvasively with continuous positive airway pressure (CPAP) to diagnose mild ARDS.

In this respect, it is noted that ARDS and Acute Lung Injury (ALI) as used herein may, as known in the art (Hernu, R., Wallet, F., Thiollibre, F. et al. An attempt to validate the modification of the American-European consensus definition of acute lung injury/acute respiratory distress syndrome by the Berlin definition in a university hospital. Intensive Care Med 39, 2161-2170 (2013)), be used interchangeably within the boundaries of the above definition, i.e., using the P/F boundaries and further criteria of the 2012 Berlin definition to distinguish between mild, moderate and severe disease.

In some aspects of the present disclosure, ARDS is classified as severe if baseline P/F ratio is ≤100 mmHg. In some aspects, ARDS is classified as moderate if baseline P/F ratio is >100 and ≤200 mmHg. In other aspects, other diagnostic measurements can be used to determine the severity of the ARDS. In some aspects of the present disclosure, AP is not administered to the subject if ARDS is determined to be mild or absent (e.g., if baseline P/F ratio is >200 mmHg).

In some aspects, an AP, e.g., RecAP, is administered prophylactically. In some aspects, the methods disclosed herein comprise administering at least one dose of AP, e.g., RecAP, to the subject wherein each dose is least 500 U/kg (0.8 mg/kg for the clinical grade RecAP used) or 1000 U/kg (1.6 mg/kg for the clinical grade RecAP used). In some specific aspects, the dosage regimen comprises administering 0.5 mg/kg to 2 mg/kg of an AP, e.g., RecAP, by intravenous infusion, in daily doses, for at least three days. In some specific aspects, the dosage regimen comprises administering 0.5 mg/kg to 2 mg/kg of an AP, e.g., RecAP, by intravenous infusion, in daily doses, for at least three days.

In order that the present disclosure can be more readily understood, certain terms are first defined. Additional definitions are set forth throughout the detailed description.

### I. Definitions

In this specification and the appended claims, the singular forms “a”, “an” and “the” include plural referents unless the context clearly dictates otherwise. The terms “a” (or “an”), as well as the terms “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Wherever aspects are described herein with the language “comprising,” otherwise analogous aspects described in terms of “consisting of” and/or “consisting essentially of” are also provided.

The term “about” as used in connection with a numerical value throughout the specification and the claims denotes an interval of accuracy, familiar and acceptable to a person skilled in the art. In general, such interval of accuracy is ±15%.

Unless defined otherwise, all technical and scientific terms used herein have the same meaning as commonly understood by one of ordinary skill in the art to which this disclosure is related.

Units, prefixes, and symbols are denoted in their Système International de Unites (SI) accepted form.

Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the invention. The lower limit of a range is excluded from the range if the value of the lower limit is preceded by a “>”. The upper limit of a range is excluded from the range if the value of the upper limit is preceded by a “<”. The lower limit of a range is included in the range if the value of the lower limit is preceded by a “≥”. The upper limit of a range is included in the range if the value of the upper limit is preceded by a “≤”. Where a value is explicitly recited, it is to be understood that values which are about the same quantity or amount as the recited value are also within the scope of the invention. Where a combination is disclosed, each subcombination of the elements of that combination is also specifically disclosed and is within the scope of the invention. Conversely, where different elements or groups of elements are individually disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

As used herein the terms “treat,” “treatment,” or “treatment of” refers to (i) reducing the potential or risk for a disease or disorder, e.g., ARDS, (ii) reducing the occurrence of a disease or disorder, e.g., ARDS, (iii) reducing the severity (e.g., ameliorating the symptoms) of a disease or disorder, e.g., ARDS, or (iv) a combination thereof.

For example, treating can refer to the ability of a therapy when administered to a subject, to prevent or reduce the risk of lung injury due to or associated with ARDS, from occurring and/or to cure or to alleviate symptoms, signs, or causes of lung injury, e.g., ARDS. Treating also refers to mitigating or decreasing at least one clinical symptom and/or inhibition or delay in the progression of the condition and/or prevention or delay of the onset of a disease or illness when compared with a non-treated (or placebo treated) group. Thus, the terms “treat,” “treating” or “treatment of” (or grammatically equivalent terms) refer to therapeutic treatment regimes for ARDS that may prevent (further) lung injury caused by or associated with ARDS. In case of respiratory disease, it is preferred that subjects, after being treated with an AP with a method as disclosed herein improve in respiratory function or less decline in respiratory function, relative to subjects that have not been treated with said AP.

As used herein, the term “preserving” includes preventing a reduction, slowing down a reduction, stopping a reduction and/or at least partly reversing a reduction of a lung function. The term “increasing” is not necessarily limited to increasing said respiratory function to a value equal to or higher than that before said treatment occurred. It includes partly restoring respiratory function.

The terms “subject” or “patient” as used herein refer to any subject, particularly a mammalian subject, for whom therapy or prognosis of lung injury, e.g., ARDS is desired. As used herein, the terms “subject” or “patient” include any human or nonhuman animal. As used herein, phrases such as “a patient having ARDS” or a “patient having sepsis” includes subjects, such as mammalian subjects, that would benefit from the administration of a therapy with AP, as disclosed herein.

In some aspects of the present disclosure, a subject is a naïve subject. A naïve subject is a subject that has not been administered a therapy, for example a therapeutic agent. In some aspects, a naïve subject has not been treated with a therapeutic agent prior to being diagnosed with lung injury, e.g., ARDS, or a disease or condition (e.g., sepsis) that can lead to lung injury.

In another aspect, a subject has received therapy and/or one or more doses of a therapeutic agent prior to being diagnosed as having lung injury, e.g., ARDS, or a disease or condition (e.g., sepsis) that can lead to lung injury and/or ARDS.

In some aspects, a subject can be administered at least one therapeutically effective dose of an AP, e.g., RecAP, if the subject's baseline P/F ratio is below a predetermined P/F ratio threshold level or if the baseline P/F ratio is within a predetermined range.

The terms “therapeutic agent” and “drug” as used herein also refer to any therapeutically active substance that is administered to a subject having a disease or disorder, e.g., lung injury, e.g., ARDS, or a disease or condition (e.g., sepsis) that can lead to lung injury to produce a desired, usually beneficial, effect. A therapeutic agent can also be a pro-drug, which metabolizes into the desired therapeutically active substance when administered to a subject. In some aspects, the therapeutic agent is a prophylactic agent. In addition, a therapeutic agent can be pharmaceutically formulated. A therapeutic agent can also be or comprise a radioactive isotope or agent activated by some other form of energy such as light or ultrasonic energy, or by other circulating molecules that can be systemically administered.

In some aspects of the present disclosure, a therapeutic agent for the treatment, prevention, or amelioration of the symptom of lung injury, e.g., ARDS, or a disease or condition (e.g., sepsis) that can lead to lung injury can comprise an AP, e.g., RecAP; alone or in combination with one or more standard therapeutic agents generally used for the treatment of lung injury, e.g., ARDS, or a disease or condition (e.g., sepsis) that can lead to lung injury.

A “therapeutically effective” amount as used herein is an amount of therapeutic agent that provides some improvement or benefit to a subject having a disease or disorder, e.g., lung injury, e.g., ARDS, or a disease or condition (e.g., sepsis) that can lead to lung injury. Thus, a “therapeutically effective” amount is an amount that provides some alleviation, mitigation, and/or decrease in at least one clinical symptom of a disease or disorder, e.g., lung injury, e.g., ARDS, or a disease or condition (e.g., sepsis) that can lead to lung injury.

Clinical symptoms associated with lung injury, e.g., ARDS, or a disease or condition (e.g., sepsis) that can lead to lung injury that can be treated by the compositions, methods, as specific dosage regimens of the disclosure are well known to those skilled in the art. Further, those skilled in the art will appreciate that the therapeutic effects need not be complete or curative, as long as some benefit is provided to the subject. In some aspects, the term “therapeutically effective” refers to an amount of a therapeutic agent that is capable of altering biomarker levels, e.g., P/F ratio in a patient in need thereof.

As used herein, a “sufficient amount” or “an amount sufficient to” achieve a particular result in a patient having a disease or disorder, e.g., lung injury, e.g., ARDS, or a disease or condition (e.g., sepsis) that can lead to lung injury refers to an amount of a therapeutic agent (e.g., an AP such as RecAP) that is effective to produce a desired effect, which is optionally a therapeutic effect (i.e., by administration of a therapeutically effective amount). In some aspects, such particular result is an improvement in lung function.

As used herein, the term “healthcare provider” refers to individuals or institutions that directly interact and administer to living subjects, e.g., human patients. Non-limiting examples of healthcare providers include doctors, nurses, technicians, therapist, pharmacists, counselors, alternative medicine practitioners, medical facilities, doctor's offices, hospitals, emergency rooms, clinics, urgent care centers, alternative medicine clinics/facilities, and any other entity providing general and/or specialized treatment, assessment, maintenance, therapy, medication, and/or advice relating to all, or any portion of, a patient's state of health, including but not limited to general medical, specialized medical, surgical, and/or any other type of treatment, assessment, maintenance, therapy, medication and/or advice.

As used herein, the term “clinical laboratory” refers to a facility for the examination or processing of materials derived from a living subject, e.g., a human being. Non-limiting examples of processing include biological, biochemical, serological, chemical, immunohematological, hematological, biophysical, cytological, pathological, genetic, or other examination of materials derived from the human body for the purpose of providing information, e.g., for the diagnosis, prevention, or treatment of any disease or impairment of, or the assessment of the health of living subjects, e.g., human beings. These examinations can also include procedures to collect or otherwise obtain a sample, prepare, determine, measure, or otherwise describe the presence or absence of various substances in the body of a living subject, e.g., a human being, or a sample obtained from the body of a living subject, e.g., a human being.

As used herein, the term “healthcare benefits provider” encompasses individual parties, organizations, or groups providing, presenting, offering, paying for in whole or in part, or being otherwise associated with giving a patient access to one or more healthcare benefits, benefit plans, health insurance, and/or healthcare expense account programs.

In some aspects, a healthcare provider can administer or instruct another healthcare provider to administer a therapy to prevent, treat, or ameliorate the symptoms of a disease or disorder, e.g., lung injury, e.g., ARDS, or a disease or condition (e.g., sepsis) that can lead to lung injury. A healthcare provider can implement or instruct another healthcare provider or patient to perform the following actions: obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, administer a therapy (e.g., an AP such as RecAP), commence the administration of a therapy, cease the administration of a therapy, continue the administration of a therapy, temporarily interrupt the administration of a therapy, increase the amount of an administered therapeutic agent, decrease the amount of an administered therapeutic agent, continue the administration of an amount of a therapeutic agent, increase the frequency of administration of a therapeutic agent, decrease the frequency of administration of a therapeutic agent, maintain the same dosing frequency on a therapeutic agent, replace a therapy or therapeutic agent by at least another therapy or therapeutic agent, combine a therapy or therapeutic agent with at least another therapy or additional therapeutic agent.

In some aspects, a healthcare benefits provider can authorize or deny, for example, collection of a sample, processing of a sample, submission of a sample, receipt of a sample, transfer of a sample, analysis or measurement a sample, quantification a sample, provision of results obtained after analyzing/measuring/quantifying a sample, transfer of results obtained after analyzing/measuring/quantifying a sample, comparison/scoring of results obtained after analyzing/measuring/quantifying one or more samples, transfer of the comparison/score from one or more samples, administration of a therapy or therapeutic agent, commencement of the administration of a therapy or therapeutic agent, cessation of the administration of a therapy or therapeutic agent, continuation of the administration of a therapy or therapeutic agent, temporary interruption of the administration of a therapy or therapeutic agent, increase of the amount of administered therapeutic agent, decrease of the amount of administered therapeutic agent, continuation of the administration of an amount of a therapeutic agent, increase in the frequency of administration of a therapeutic agent, decrease in the frequency of administration of a therapeutic agent, maintain the same dosing frequency on a therapeutic agent, replace a therapy or therapeutic agent by at least another therapy or therapeutic agent, or combine a therapy or therapeutic agent with at least another therapy or additional therapeutic agent.

In addition a healthcare benefits provider can, e.g., authorize or deny the prescription of a therapy, authorize or deny coverage for therapy, authorize or deny reimbursement for the cost of therapy, determine or deny eligibility for therapy, etc.

In some aspects, a clinical laboratory can, for example, collect or obtain a sample, process a sample, submit a sample, receive a sample, transfer a sample, analyze or measure a sample, quantify a sample, provide the results obtained after analyzing/measuring/quantifying a sample, receive the results obtained after analyzing/measuring/quantifying a sample, compare/score the results obtained after analyzing/measuring/quantifying one or more samples, provide the comparison/score from one or more samples, obtain the comparison/score from one or more samples, or other related activities.

### II. Treatment of ARDS with AP

In certain aspects, the present disclosure is related to methods for preserving or improving lung function in populations of subjects, in particular ARDS patients, that have been determined to respond particularly well to treatment with AP.

Recent analysis of the data from the STOP-AKI study, which has been largely described in Pickkers et al (Pickkers P, Mehta R L, Murray P T, et al. Effect of Human Recombinant Alkaline Phosphatase on 7-Day Creatinine Clearance in Patients With Sepsis-Associated Acute Kidney Injury: A Randomized Clinical Trial. JAMA. 2018; 320(19):1998-2009) has identified statistically significant correlations between thresholds corresponding to degrees of severity of ARDS and improvements in lung function (as evidenced by improved P/F ratio with respect to placebo). Stratifying patients in the STOP-AKI clinical trial according to, e.g., markers of lung function, in particular P/F ratio has surprisingly identified particular effects of AP administration on specific subgroups. The parameter or parameters defining each of these subgroups (e.g., a series of thresholds, such as P/F ratio thresholds) can be used, e.g., to personalize AP therapy to specific subgroups, to selected patients for treatment, to make decisions related to the AP treatment (e.g., modify dosing or dosage schedule), or to evaluate the likelihood of a positive outcome.

Accordingly, in some aspects, the methods disclosed herein relate to the administration of AP, e.g., RecAP, to a subject determined to have either moderate or severe ARDS, comprising administering an AP such as RecAP to the subject. The skilled person is well aware how to classify a subject's ARDS severity as being moderate or severe. As described above, the Berlin 2012 definition classifies moderate ARDS as having a P/F of >100 and ≤200 mmHg and severe ARDS as having a P/F≤100 mmHg (≤13.33 kPa). A person having moderate or severe ARDS thus has a P/F ratio of ≤200 mmHg, The Berlin definition further requires a minimum positive end expiratory pressure (PEEP) of 5 cmH₂O for consideration of the P/F ratio. In a preferred embodiment, therefore, the invention provides an alkaline phosphatase (AP) for use in a method for treating acute respiratory distress syndrome (ARDS) in a subject in need thereof, wherein the subject has moderate or severe ARDS with a P/F ratio of ≤200 mmHg, if said P/F ratio is measured using a minimum positive end expiratory pressure (PEEP) of 5 cmH₂O. Administration of AP is particularly effective when administered to subjects having moderate to severe ARDS at doses of least 500 U/kg. AP is particularly effective when administered at doses of at least 1,000 U/kg.

In some aspects, the AP (e.g., RecAP) is administered as doses of at least about 500 U/kg, at least about 600 U/kg, at least about 700 U/kg, at least about 800 U/kg, at least about 900 U/kg, at least about 1000 U/kg, at least about 1100 U/kg, at least about 1200 U/kg, at least about 1300 U/kg, at least about 1400 U/kg, at least about 1500 U/kg, at least about 1600 U/kg, at least about 1700 U/kg, at least about 1800 U/kg, at least about 1900 U/kg, or at least about 2000 U/kg per dose. In some aspects, the AP (e.g. RecAP) is administered as doses above 2000 U/kg per dose. In some aspects, the AP (e.g., RecAP) is administered as doses below 500 U/kg per dose.

In some aspects, the AP (e.g., RecAP) is administered at a dose between about 500 U/kg and about 1500 U/kg, between about 600 U/kg and about 1400 U/kg, between about 700 U/kg and about 1300 U/kg, between about 800 U/kg and about 1200 U/kg, or between about 900 U/kg and about 1100 U/kg. In some specific aspects, AP is administered as about 1000 U/kg doses.

In some aspects, the AP is a human AP. In some aspects, the AP is a recombinant AP. In some aspects, the AP is a chimeric AP. In a particular aspects, the chimeric AP is RecAP (SEQ ID NO: 1). In some aspects, an AP disclosed herein has at least about 70%, at least about 75%, at least about 80%, at least about 85%, at least about 90%, at least about 95%, at least about 96%, at least about 97%, at least about 98%, or at least about 99% sequence identity to the amino acid sequence of SEQ ID NO:1. In some aspects, the AP is a functional fragment (i.e., a fragment of the AP, e.g., AP conserving at least about 10%, at least about 20%, at least about 30%, at least 40%, at least about 50%, at least about 60%, at least 70%, at least about 80%, or at least about 90% of the AP activity of the corresponding full length AP). In some aspects, the AP is a variant or a derivative of an AP disclosed herein. Other AP that can be used as disclosed herein are discussed in detail below.

In some aspects, the AP is RecAP (e.g., the clinical grade RecAP used in the present disclosure), and it is administered at a dose of at least about 0.1 mg/kg, at least about 0.2 mg/kg, at least about 0.3 mg/kg, at least about 0.4 mg/kg, at least about 0.5 mg/kg, at least about 0.6 mg/kg, at least about 0.7 mg/kg, at least about 0.8 mg/kg, at least about 0.9 mg/kg, at least about 1 mg/kg, at least about 1.1 mg/kg, at least about 1.3 mg/kg, at least about 1.4 mg/kg, at least about 1.5 mg/kg, at least about 1.6 mg/kg, at least about 1.7 mg/kg, at least about 1.8 mg/kg, at least about 1.9 mg/kg, at least about 2 mg/kg, at least about 2.1 mg/kg, at least about 2.2 mg/kg, at least about 2.3 mg/kg, or at least about 2.4/kg per dose. In some aspects, the AP is administered as doses above 2.4 mg/kg per dose. In some aspects, the AP is RecAP (e.g., the clinical grade RecAP used in the present disclosure), and it is administered at a dose of at least about 100 U/kg, at least about 200 U/kg, at least about 300 U/kg, at least about 400 U/kg, at least about 500 U/kg, at least about 600 U/kg, at least about 700 U/kg, at least about 800 U/kg, at least about 900 U/kg, at least about 1000 U/kg, at least about 1100 U/kg, at least about 1200 U/kg, at least about 1300 U/kg, at least about 1400 U/kg, at least about 1500 U/kg, at least about 1600 U/kg, at least about 1700 U/kg, at least about 1800 U/kg, at least about 1900 U/kg, or at least about 2000 U/kg., the AP is RecAP, and it is administered at a dose below 100 U/kg., the AP is RecAP, and it is administered at a dose above 2000 U/kg.

In some aspects, the AP is RecAP (e.g., the clinical grade RecAP used in the present disclosure) and it is administered at a dose between about 0.8 mg/kg and about 2.4 mg/kg, between about 0.9 mg/kg and about 2.3 mg/kg, between about 1 mg/kg and about 2.2 mg/kg, between about 1.1 mg/kg and about 2.1 mg/kg, between about 1.2 mg/kg and about 2 mg/kg, between about 1.3 mg/kg and about 1.9 mg/kg, between about 1.4 mg/kg and about 1.8 mg/kg, or between about 1.5 mg/kg and about 1.7 mg/kg. In some specific aspects, AP is administered as about 1.6 mg/kg doses.

In some aspects, the AP is RecAP (e.g., the clinical grade RecAP used in the present disclosure) and it has a specific activity of at least about 100 U/mg, at least about 200 U/mg, at least about 300 U/mg, at least about 400 U/mg, at least about 500 U/mg, at least about 600 U/mg, at least about 700 U/mg, at least about 800 U/mg, at least about 900 U/mg, at least about 1000 U/mg, at least about 1100 U/mg, at least about 1200 U/mg, at least about 1300 U/mg, at least about 1400 U/mg, at least about 1500 U/mg, at least about 1600 U/mg, at least about 1700 U/mg, at least about 1800 U/mg, at least about 1900 U/mg, or at least about 2000 U/mg.

In some aspects, the AP is RecAP (e.g., the clinical grade RecAP used in the present disclosure) and it has a specific activity of about 1000 U:1.6 mg. In some aspects, the AP is RecAP and it has a specific activity between about 600 U/mg and about 700 U/mg, or between about 500 U/mg and about 800 U/mg, or between about 400 U/mg and about 900 U/mg, or between about 300 U/mg and about 1000 U/mg, or between about 200 U/mg and about 1100 U/mg, or between 100 U/mg and about 1200 U/mg. In some aspects, the AP is RecAP and it has a specific activity below 100 U/mg. In some aspects, the AP is RecAP and it has a specific activity above 1200 U/mg.

In some aspects, only one dose of AP (e.g., RecAP) is administered per treatment (e.g., one dose per day for 1-7 days). In other aspects, more than one dose of AP is administered. In some aspects, two, three, four, five, six, seven, eight, nine, 10, 11, 12, 13, 14, 15, 16, 17, 18, 19, 20, or 21 doses of AP are administered (e.g., at least two doses per day for 1-7 days).

In some aspects, the AP doses are administered daily. In other aspects, AP doses are administered every 2, 3, 4, 5, 6 or 7 days.

In some aspects, a single dose is administered every day. In some aspects, 2, 3, or more doses are administered every day.

In some aspects, the treatment with AP is less than about 4 days. In some aspects, the treatment with AP is less than 3 days, less than 2 days, or less than 1 day.

In a particular aspect, the AP is administered as a daily about 1000 U/kg dose administered in 3 consecutive days. In some particular aspects, when the AP is RecAP, the AP is administered as daily 1.6 mg/kg doses administered in 3 consecutive days. In some aspects, each AP, e.g., RecAP (e.g., the clinical grade RecAP used in the present disclosure), dose is between about 0.10 mg/kg and about 3 mg/kg, or between about 0.20 mg/kg and about 2.9 mg/kg, or between about 0.3 mg/kg and about 2.8 mg/kg, or between about 0.4 mg/kg and about 2.7 mg/kg, or between about 0.5 mg/kg and about 2.6 mg/kg, or between about 0.6 mg/kg and about 2.5 mg/kg, or between about 0.7 mg/kg and about 2.4 mg/kg, or between about 0.8 mg/kg and about 2.3 mg/kg, or between about 0.9 mg/kg and about 2.2 mg/kg, or between about 1 mg and about 2.1 mg/kg, or between about 1.1 mg/kg and about 2 mg/kg, or between about 1.2 mg/kg and about 1.9 mg/kg, or between about 1.3 mg/kg and about 1.8 mg/kg, or between about 1.4 mg/kg and about 1.7 mg/kg. In some aspects, each AP, e.g., RecAP, dose comprises at least about 0.1 mg AP/kg, at least about 0.2 mg AP/kg, at least about 0.3 mg AP/kg, at least about 0.4 mg AP/kg, at least about 0.5 mg AP/kg, at least about 0.6 mg AP/kg, at least about 0.7 mg AP/kg, at least about 0.8 mg AP/kg, at least about 0.9 mg AP/kg, at least about 1 mg AP/kg, at least about 1.1 mg AP/kg, at least about 1.2 mg AP/kg, at least about 1.3 mg AP/kg, at least about 1.4 mg AP/kg, at least about 1.5 mg AP/kg, at least about 1.6 mg AP/kg, at least about 1.7 mg AP/kg, at least about 1.8 mg AP/kg, at least about 1.9 mg AP/kg, at least about 2 mg AP/kg, at least about 2.1 mg AP/kg, at least about 2.2 mg AP/kg, at least about 2.3 mg AP/kg, at least about 2.4 mg AP/kg, at least about 2.5 mg AP/kg, at least about 2.6 mg AP/kg, at least about 2.6 mg AP/kg, at least about 2.7 mg AP/kg, at least about 2.8 mg AP/kg, at least about 2.9 mg AP/kg, or at least about 3 mg AP/kg.

The AP may be administered via different routes, for example intravenously, rectally, bronchially or orally. In some specific aspects, the AP is administered intravenously, e.g., via intravenous infusion. In some aspects, AP is administered intravenously via continuous infusion.

Although short term preservation of lung function can have immediate life-saving consequences, it is preferred that the effect of AP on the lung function is long lasting.

P/F ratio can be determined by methods known in the art. The arterial pO₂ measured by arterial blood gas (ABG) is the preferred method for calculating the P/F ratio. However, when the pO₂ is unknown because an ABG is not available, the SpO₂ measured by pulse oximetry can be used to approximate the pO₂, as shown in the Table below. It is important to note that estimating the pO₂ from the SpO₂ becomes unreliable when the SpO₂ is 98%-100%.

| SpO₂ (%) | 86 | 87 | 88 | 89 | 90 | 91 | 92 | 93 | 94 | 95 | 96 | 97 |
|---|---|---|---|---|---|---|---|---|---|---|---|---|
| PO₂ (mmHg) | 51 | 52 | 54 | 56 | 58 | 60 | 64 | 68 | 73 | 80 | 90 | 110 |

Example: Suppose a patient on 40% oxygen has a pulse oximetry SpO2 of 95%. Referring to the Table above, SpO2 of 95% is equal to a pO2 of 80 mmHg. The P/F ratio=80 divided by 0.40=200. The patient may be stable receiving 40% oxygen, but still has acute respiratory failure. If oxygen were withdrawn leaving the patient on room air, the pO2 would only be 40 mmHg (much less than the cut-off value for acute respiratory failure of 60 mmHg on room air).

Supplemental oxygen may be administered either by mask or by nasal cannula (“NC”). A Venturi mask (Venti-mask) delivers a controlled flow of oxygen at a specific fixed concentration (FIO₂): 240, 28%, 31%, 35%, 40%, or 50%. The non-rebreather (“NRB”) mask is designed to deliver approximately 100% oxygen. Providing 40% or more supplemental oxygen implies that the physician is treating acute respiratory failure since only a patient with acute respiratory failure would need that much oxygen. A nasal cannula provides oxygen at adjustable flow rates in liters of oxygen per minute (L/min or “LPM”). The actual FIO₂ (percent oxygen) delivered by nasal cannula is somewhat variable and less reliable than with a mask, but can be estimated as shown in the Table below. The FIO₂ derived from nasal cannula flow rates can then be used to calculate the P/F ratio.

| Flow rate (L/min) | 1 | 2 | 3 | 4 | 5 | 6 |
|---|---|---|---|---|---|---|
| FIO₂ (%) | 24 | 28 | 32 | 36 | 40 | 44 |

Example: A patient has a pO2 of 85 mmHg on ABG while receiving 5 L/min of oxygen. Since 5 L/min is equal to 40% oxygen (an FIO₂ of 0.40), the P/F ratio=85 divided by 0.40=212.5.

In some aspects, the administration of AP to the subject as disclosed herein is capable of improving many respiratory function related parameters, such as P/F ratio, oxygen saturations in arterial blood (pO₂), pulse oxygen saturation (SpO₂), oxygenation index, and/or respiratory index in the subject.

In some aspects, the administration of AP (e.g., RecAP) to the subject results in an increase in P/F ratio with respect to the P/F ratio of the subject in the absence of treatment. In some aspects, the increase in P/F ratio is at least 5, at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, or at least 200 mmHg with respect to the P/F ratio of the subject in the absence of treatment.

In some aspects, the administration of AP (e.g., RecAP) to the subject results in an increase in the subject's P/F ratio with respect to a baseline P/F ratio. In some aspects, the increase in P/F ratio of the subject is at least 5, at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200 mmHg with respect to the baseline P/F ratio. In some aspects, the administration of AP (e.g., RecAP) to an ARDS patient results in an attenuation in severity of ARDS from “severe” to “moderate”, or from “moderate” to “mild”, or from “severe” to “mild”, or from “moderate” or “severe” to “no ARDS”, i.e. P/F >300 mmHg.

Within this context, baseline P/F ratio is defined as the P/F ratio before or around the start of the treatment. Baseline P/F ratio is preferably determined at day −2, day −1, day 0, or day 1, i.e., preferably within 48 hours before start of treatment with AP and up to 24 hours after the start of treatment with AP. More preferably, Baseline P/F ratio is determined within 24 hours before start of treatment with AP and up to 12 hours after the start of treatment with AP, more preferably between 12 hours before the start of the treatment with AP and up to 6 hours after the start of treatment with AP, more preferably between 12 hours before the start of the treatment with AP and up to the start of treatment with AP, most preferably between 6 hours before the start of the treatment with AP and up to the start of treatment with AP. AP treatment is preferably continued until the P/F ratio is at least 200 or more, more preferably at least 300 or more, most preferably until the subject is considered not to have ARDS anymore.

In some aspects, the increase in lung function, e.g., increase in P/F ratio, is observed 1, 2, 3, 4, 5, 6, 7, 8, 9, 10, 11, 12, 13, 14, 15, 16, 17, 18, 19, 20, 21, 22, 23, 24, 25, 26, 27, or 28 days after AP administration.

In some aspects of the present disclosure, ARDS of the subject is accompanied by or due to sepsis or a viral infection. In some aspects, ARDS of the subject is accompanied by or due to sepsis or a viral infection, and AP (e.g., RecAP) is administered to the subject only if sepsis or a viral infection is determined less than 96 hours prior to the decision to initiate the treatment. In other aspects, ARDS of the subject is accompanied by or due to sepsis or a viral infection and AP (e.g., RecAP) is administered to the subject only if sepsis or a viral infection is determined less than 72 hours prior to ARDS detection.

In some aspects of the present disclosure, ARDS of the subject is accompanied by or due to sepsis or a viral infection and treatment of the subject with AP (e.g., RecAP) is initiated within 24 hours after sepsis or a viral infection is determined. The presence of sepsis can be detected, e.g., by using criteria developed for determining sepsis, such SIRS or SOFA. The definition of “sepsis” has been reassessed several times since it was first defined in 1992, focusing on the then-prevailing view that sepsis resulted from a host's systemic inflammatory response syndrome (SIRS) to infection (Bone R C, Balk R A, Cerra F B, et al. American College of Chest Physicians/Society of Critical Care Medicine Consensus Conference: definitions for sepsis and organ failure and guidelines for the use of innovative therapies in sepsis. Crit Care Med. 1992; 20(6):864-874). In 2001, the list of diagnostic criteria was expanded, but no alternative criteria were provided (Levy M M, Fink M P, Marshall J C, et al. International Sepsis Definitions Conference. 2001 SCCM/ESICM/ACCP/ATS/SIS International Sepsis Definitions Conference. Intensive Care Med. 2003; 29(4):530-538). In 2016, the now widely used “SEPSIS-3” criteria were recommended, that no longer make use of the SIRS criteria. Instead Sequential [Sepsis-related] Organ Failure Assessment (SOFA) is used to diagnose sepsis (Singer M, Deutschman C S, Seymour C W, et al. The Third International Consensus Definitions for Sepsis and Septic Shock (Sepsis-3). JAMA. 2016; 315(8):801-810. doi:10.1001/jama.2016.0287). The STOP-AKI study, as described in the Examples, was conducted between 2014 and 2017 and made use of both SIRS and SOFA score to identify and/or stratify sepsis patients. For the definition and determination of “sepsis” as used herein, the SEPSIS-3 criteria, based on SOFA score and extensively described in Singer et al (Singer M, Deutschman C S, Seymour C W, et al. The Third International Consensus Definitions for Sepsis and Septic Shock (Sepsis-3). JAMA. 2016; 315(8):801-810. doi:10.1001/jama.2016.0287) are preferably used.

In some aspects of the present disclosure, AP is administered to a subject at risk of sepsis in order to prevent reduction in lung function.

In some aspects, AP treatment of the subject is initiated within 48 hours after ARDS is detected. In a preferred embodiment, AP treatment of the subject is initiated within 48 hours, preferably within 24 hours, more preferably within 12 hours, most preferably within 6 hours after moderate or severe ARDS is detected.

In some aspects of the methods disclosed herein, the administration of at least one dose of AP results in a shortening of duration or cessation of mechanical ventilation therapy in a subject undergoing mechanical ventilation therapy.

In some aspects of the methods disclosed herein, the administration of AP results in an increase of lung function or prevents the reduction of lung function below a critical threshold. Accordingly, in some aspects, AP administration is able to prevent reduction of the lung function below a critical threshold. Thus, in some aspects, an indicator of lung function (e.g., P/F ratio) is determined prior to administering AP for preserving lung function in order to determine the risk that the lung function of said person is reduced below a certain threshold level.

In some aspects, the methods disclosed herein comprise detecting changes in markers of lung function, e.g., P/F ratio, alone or in combination with the detection of changes in the levels of one, two, three, or more biomarkers.

In some aspects, the methods disclosed herein comprise predicting an increased clinical response to therapy with AP, e.g., RecAP, based on detected lung function parameters (e.g., baseline P/F ratio). In some aspects, the methods of the present disclosure comprise evaluating whether a lung function parameter (e.g., baseline P/F ratio) falls within a certain range, or whether it is above or below a certain threshold (e.g., P/F ratio threshold for severity of ARDS). Thus, if, e.g., the lung function parameter (e.g., baseline P/F ratio), alone or in combination with other biomarkers, indicates that the patient will benefit from therapy with AP, then therapy could commence, or be maintained, or be modified (e.g., increasing or decreasing dosage, or increasing or decreasing frequency of doses).

Conversely, if, e.g., the lung function parameter (e.g., baseline P/F ratio), alone or in combination with other biomarkers, indicates that the patient will not benefit from therapy with AP, then therapy could be discontinued, temporarily suspended, modified (e.g., increasing or decreasing dosage or increasing or decreasing frequency of doses), etc.

In other words, specific levels of a lung function parameter (e.g., baseline P/F ratio) alone or in combination with other molecular or clinical biomarkers are correlated with clinical efficacy of AP therapy and useful to predict clinical outcomes in specific populations of patients suffering from sepsis and/or ARDS.

For some jurisdictions, the invention provides alkaline phosphatase (AP) for use in a method for treating acute respiratory distress syndrome (ARDS) in a subject in need thereof, wherein the subject has a P/F ratio of ≤200 mmHg. Further provided is a use of an alkaline phosphatase (AP) for the manufacture of a medicament for the treatment of acute respiratory distress syndrome (ARDS) in a subject in need thereof, wherein the subject has a P/F ratio of ≤200 mmHg.

The invention also provides alkaline phosphatase (AP) for use in a method to treat acute respiratory syndrome (ARDS) in a subject in need thereof, the method comprising administering an effective amount of alkaline phosphatase (AP) to said subject, wherein
(i) the subject has moderate or severe ARDS prior to the treatment with AP, and
(ii) the AP is to be administered in at least one 300 U/kg to 2,000 U/kg dose.

In moderate or severe ARDS, according to the Berlin 2012 definition, the P/F ratio is ≤200 mmHg when measured using a minimum positive end expiratory pressure (PEEP) of 5 cmH₂O.

In a preferred embodiment, the subject has moderate ARDS with a P/F ratio of >100 and ≤200 mmHg prior to the treatment with AP. In another preferred embodiment, the subject has severe ARDS with a P/F ratio of <100 mmHg prior to the treatment with AP.

In some preferred embodiments, an AP for use according to the invention or a use according to the invention is provided, wherein the AP is a human AP.

In some preferred embodiments, the AP is a recombinant AP, preferably a chimeric AP, more preferably an AP having at least 80%, at least 85%, at least 90%, at least 95%, at least 96%, at least 97%, at least 98%, at least 99%, or 100% sequence identity to the amino acid sequence of RecAP (SEQ ID NO: 1). In some preferred embodiments, the AP is a recombinant AP having at least 80%, at least 85%, at least 90%, at least 95%, at least 96%, at least 97%, at least 98%, at least 99%, or 100% sequence identity to the amino acid sequence of RecAP (SEQ ID NO: 1), with the proviso that the amino acid at position 279 is leucine (L), the amino acid at position 328 is valine (V) and the amino acid at position 478 is leucine (L).

In a preferred embodiment, an AP for use according to the invention or a use according to the invention is provided, wherein the administration of AP leads to an increase in lung function of the subject. Said increase in lung function preferably comprises an increase in the P/F ratio of the subject, with respect to the P/F ratio of the subject in the absence of treatment.

In some preferred embodiments, an AP for use according to the invention or a use according to the invention is provided, wherein the ARDS is associated with or due to sepsis. Preferably, the sepsis is detected less than 96 hours prior to AP administration.

In more preferred embodiments, an AP for use according to the invention or a use according to the invention is provided, wherein the ARDS is associated with or due to sepsis, and the sepsis is detected less than 72 hours prior to ARDS detection. Preferably, said AP treatment is initiated within 24 hours after sepsis is detected and/or after ARDS is detected.

In other preferred embodiments, an AP for use according to the invention or a use according to the invention is provided, wherein the ARDS is associated with or due to a virus infection. Said virus infection preferably comprises a coronaviral virus infection, more preferably an infection with a severe acute respiratory syndrome (SARS)-related coronavirus. In some preferred embodiments, said coronavirus is SARS-CoV-2.

In a preferred embodiment, an AP for use according to the invention or a use according to the invention is provided, wherein the AP is administered once daily. In a preferred embodiment, the AP is administered intravenously. In a preferred embodiment, the AP is administered in three daily doses.

In some preferred embodiments, AP for use according to the invention or a use according to the invention is provided, wherein the AP is RecAP and the dose is between 0.6 mg/kg (or 375 U/kg) and 3.2 mg/kg (or 2,000 U/kg) of RecAP, preferably between 0.8 mg/kg (or 500 U/kg) and 2.0 mg/kg (or 1250 U/kg), more preferably about 1.6 mg/kg (or 1000 U/kg).

Some preferred embodiments provide an AP for use according to the invention, or a use according to the invention, wherein the AP is to be administered in at least one 500 U/kg to 2,000 U/kg dose. Preferably, the AP dose is 500 U/kg or 1000 U/kg of RecAP. In some preferred embodiments, the AP dose id 0.8 mg/kg or 1.6 mg/kg of RecAP.

In a preferred embodiment, AP for use according to the invention or a use according to the invention is provided, wherein the administration of at least one dose of AP results in the preservation or increase of the P/F ratio in the subject. Preferably, the administration of at least one dose of AP results in a shortening of duration or cessation of mechanical ventilation therapy in a subject undergoing mechanical ventilation therapy.

The term “biomarker” as used herein refers to a factor that is a distinctive indicator of a biological process, biological event, and/or pathologic condition, e.g., a predictor of clinical response to treatment with AP, e.g., RecAP. As used herein, the term biomarker encompasses both clinical markers and molecular biomarkers (biological markers). Thus, in the context of the present disclosure, the term “biomarker” encompasses, e.g., “biological biomarkers” or “molecular biomarkers.” In some aspects, the biological or molecular biomarkers used to evaluate lung function comprise markers of inflammation, e.g., C-reactive protein (CRP), fibrinogen, interleukin 6 (IL-6), or interleukin 8 (IL-8). More specific biomarkers that are associated with diagnosis and outcome of ARDS are receptor for advanced glycation end-products (RAGE), angiopoietin-2 (Ang-2), surfactant protein D (SP-D), inteleukin-8, Fas and Fas ligand, procollagen peptide (PCP) I and III, octane, acetaldehyde, and 3-methylheptane. In general, these are cell-specific for epithelial or endothelial injury or involved in the inflammatory or infectious response (Garcia-Laorden M I, Lorente J A, Flores C, Slutsky A S, Villar J. Biomarkers for the acute respiratory distress syndrome: how to make the diagnosis more precise. Ann Transl Med. 2017; 5(14):283).

As disclosed above, the term “biomarker” also encompasses “clinical biomarkers,” also referred to as “clinical status markers,” that can be predictive of response to biological therapies, for example, gender, age, concomitant drugs, smoking status, body mass index (BMI), etc.

As discussed above, a cut-off approach based on baseline P/F values is applied to classify ARDS as “severe ARDS”, or “moderate ARDS”. The differences in P/F levels observed, e.g., in subjects with “severe ARDS,” “moderate to severe ARDS,” or “mild ARDS,” can be applied to predicting clinical outcomes when the patients are treated with an AP, for example, RecAP. Thus, if a subject's P/F ratio is below a certain threshold (e.g., 200 mmHg), that subject would become a candidate for treatment with a certain AP therapy, e.g., therapy with a certain AP regimen comprising one or more doses of RecAP.

In some aspects, the mere determination that the P/F ratio is below a predetermined threshold level would suffice to identify a subject as a candidate for treatment with a certain AP therapy, e.g., therapy with RecAP. Thus, in some aspects of the methods disclosed herein, P/F rates can be used alone. However, in other aspects, the P/F rate can be combined with other measures of lung function (e.g., using spirometry) and/or molecular or clinical biomarkers, such as for instance a marker selected from the group consisting of C-reactive protein (CRP), fibrinogen, interleukin 6 (IL-6), or interleukin 8 (IL-8), receptor for advanced glycation end-products (RAGE), angiopoietin-2 (Ang-2), surfactant protein D (SP-D), inteleukin-8, Fas and Fas ligand, procollagen peptide (PCP) I and III, octane, acetaldehyde, and 3-methylheptane.

These findings can be applied, for example, to devising new methods of determining treatment (e.g., by selecting patients as candidates for a certain AP therapy), methods of treating a decrease in lung function, preventing a decrease in lung function, increasing lung function, or preserving lung function (e.g., to treat ARDS), methods of monitoring efficacy of an AP treatment, or methods to adjust formulations, dosage regimens, or routes of administration.

The methods disclosed herein include prescribing, initiating, and/or altering prophylaxis and/or treatment, based on severity of ARDS, as typically categorized based on a subject's P/F ratio as described above) alone, or in combination with one or more additional biomarkers.

The present disclosure provides a method of determining whether to treat a patient having ARDS with a therapeutic regimen comprising the administration of an AP wherein the method comprises: (a) determining P/F ratio (or another lung function parameter), and, optionally, measuring or instructing a clinical laboratory to measure levels of additional biomarkers such as for instance C-reactive protein (CRP), fibrinogen, interleukin 6 (IL-6), or interleukin 8 (IL-8), receptor for advanced glycation end-products (RAGE), angiopoietin-2 (Ang-2), surfactant protein D (SP-D), interleukin-8, Fas and Fas ligand, procollagen peptide (PCP) I and III, octane, acetaldehyde, and/or 3-methylheptane, in a sample taken from the patient, and (b) treating or instructing a healthcare provider to treat the patient, or suspending the treatment, not initiating the treatment, denying the treatment, or instructing a healthcare provider to suspend, not initiate, or deny the treatment with a therapeutic regimen comprising the administration of an AP, e.g., RecAP, if the patient is determined to have a higher or lower P/F ratio (or another lung function parameter) as compared to a predetermined threshold level, and/or compared to the P/F ratio or another lung function parameter in a control, and, optionally, higher of lower levels of additional biomarkers such as for instance C-reactive protein (CRP), fibrinogen, interleukin 6 (IL-6), or interleukin 8 (IL-8), receptor for advanced glycation end-products (RAGE), angiopoietin-2 (Ang-2), surfactant protein D (SP-D), inteleukin-8, Fas and Fas ligand, procollagen peptide (PCP) I and III, octane, acetaldehyde, and/or 3-methylheptane in the sample, compared to each biomarker predetermined threshold level or levels, or compared to each biomarker level or levels in one or more controls.

In one aspect, the disclosure provides a method of determining whether to treat a patient having ARDS with a therapeutic regimen comprising the administration of an AP wherein the method comprises: (a) determining P/F ratio (or another lung function parameter) and, optionally, measuring or instructing a clinical laboratory to measure levels of additional biomarkers such as for instance C-reactive protein (CRP), fibrinogen, interleukin 6 (IL-6), or interleukin 8 (IL-8), receptor for advanced glycation end-products (RAGE), angiopoietin-2 (Ang-2), surfactant protein D (SP-D), inteleukin-8, Fas and Fas ligand, procollagen peptide (PCP) I and III, octane, acetaldehyde, and/or 3-methylheptane in a sample taken from the patient, and (b) treating or instructing a healthcare provider to treat the patient with a therapeutic regimen comprising the administration of an AP if the patient is determined to have lower or decreased P/F ratio (or another lung function parameter) as compared to a predetermined threshold level, and/or compared to the P/F ratio or another lung function parameter in a control, and, optionally, higher or lower levels of at least one of the optional additional biomarkers in the sample compared to a predetermined biomarker threshold level or levels, or compared to a biomarker level or levels in one or more controls.

In one aspect, the disclosure provides a method of determining whether to treat a patient having ARDS with a therapeutic regimen comprising the administration of an AP wherein the method comprises (a) determining the P/F ratio (or another lung function parameter) and optionally measuring or instructing a clinical laboratory to measure levels of additional biomarkers such as for instance C-reactive protein (CRP), fibrinogen, interleukin 6 (IL-6), or interleukin 8 (IL-8), receptor for advanced glycation end-products (RAGE), angiopoietin-2 (Ang-2), surfactant protein D (SP-D), inteleukin-8, Fas and Fas ligand, procollagen peptide (PCP) I and III, octane, acetaldehyde, and/or 3-methylheptane in a sample taken from the patient, and (b) suspending the treatment, not initiating treatment, denying the treatment, or instructing a healthcare provider to suspend, not initiate, or deny the treatment of the patient with a therapeutic regimen comprising the administration of an AP, e.g., RecAP, to the patient if the patient is determined to have higher or increased P/F ratio (or another lung function parameter) as compared to a predetermined threshold level, and/or compared to the P/F ratio or another lung function parameter in a control, and, optionally, higher or lower levels of the at least one optional additional biomarker in the sample compared to a predetermined biomarker threshold level or levels, or compared to a biomarker level or levels in one or more controls.

Also provided is a method of selecting a patient diagnosed with ARDS as a candidate for treatment with an AP, comprising (a) determining P/F ratio (or another lung function parameter) and, optionally, measuring or instructing a clinical laboratory to measure levels of additional biomarkers such as for instance C-reactive protein (CRP), fibrinogen, interleukin 6 (IL-6), or interleukin 8 (IL-8), receptor for advanced glycation end-products (RAGE), angiopoietin-2 (Ang-2), surfactant protein D (SP-D), inteleukin-8, Fas and Fas ligand, procollagen peptide (PCP) I and III, octane, acetaldehyde, and/or 3-methylheptanein a sample taken from the patient, and (b) treating or instructing a healthcare provider to treat the patient with an AP if the patient is determined to have lower or decreased P/F ratio (or another lung function parameter) as compared to a predetermined threshold level, and/or compared to the P/F ratio or another lung function parameter in a control, and, optionally, higher or lower levels of the at least one optional additional biomarker in the sample, compared to a predetermined threshold level or levels, or compared to a biomarker level or levels in one or more controls.

Also provided is a method of selecting a patient diagnosed with ARDS as a candidate for treatment with an AP comprising (a) determining P/F ratio (or another lung function parameter) and optionally measuring or instructing a clinical laboratory to measure levels of additional biomarkers such as for instance C-reactive protein (CRP), fibrinogen, interleukin 6 (IL-6), or interleukin 8 (IL-8), receptor for advanced glycation end-products (RAGE), angiopoietin-2 (Ang-2), surfactant protein D (SP-D), inteleukin-8, Fas and Fas ligand, procollagen peptide (PCP) I and III, octane, acetaldehyde, and/or 3-methylheptane in a sample taken from the patient, and (b) suspending the treatment, not initiating treatment, denying the treatment, or instructing a healthcare provider to suspend, not initiate, or deny the treatment of the patient with an AP, e.g., RecAP, to the patient if the patient is determined to have higher or increased P/F ratio (or another lung function parameter) as compared to a predetermined threshold level, and/or compared to the P/F ratio or another lung function parameter in a control, and, optionally, lower or decreased levels of the at least one optional additional biomarker in the sample compared to a predetermined threshold level or levels, or compared to a biomarker level or levels in one or more controls.

In some aspects, the methods disclosed can entail ordering and/or performing one or more additional assays. For example, the P/F ratio (or another lung function parameter) determination assay may be repeated to rule out a false negative result, and/or one or more additional P/F ratio (or another lung function parameter) determination assays may be performed to monitor the subject's status. Conversely, it may be desirable to repeat the P/F ratio (or another lung function parameter) determination assay to rule out a false positive result.

In some aspects, the presence of a P/F ratio (or another lung function parameter) above or below a predetermined threshold level in a patient with ARDS can be used in combination with one or more of clinical or molecular biomarkers specific for sepsis, or for the specific infection that lead to sepsis in a patient suffering from ARDS accompanied by or due to sepsis.

A person skilled in the art would understand that a P/F ratio (or another lung function parameter) can be used according to the methods disclosed herein, including but not limited to treatment, diagnostic, and monitoring methods, as a positive selector, i.e., a specific action would be taken (e.g., treating a patient) if the P/F ratio (or another lung function parameter) in the patient is below or above a predetermined P/F ratio (or another lung function parameter) threshold level, or if the P/F ratio (or another lung function parameter) is increased or decreased relative to the P/F ratio (or another lung function parameter) in one or more controls.

A person skilled in the art would understand that a P/F ratio (or another lung function parameter) can be used according to the methods disclosed herein, including but not limited to treatment, diagnostic, and monitoring methods, as a negative selector, i.e., a specific action would not be taken (e.g., treating a patient) if the P/F ratio (or another lung function parameter) in the patient is below or above a predetermined P/F ratio (or another lung function parameter) threshold level, or if the P/F ratio (or another lung function parameter) is increased or decreased relative to the P/F ratio (or another lung function parameter) in one or more controls.

In one aspect, the disclosure includes methods to facilitate a determination by a healthcare provider, a healthcare benefits provider, or a clinical laboratory to as to whether a patient will benefit from treatment with an AP.

In one aspect, the methods disclosed herein include making a diagnosis, which may be a differential diagnosis, based at least in part on P/F ratio (or another lung function parameter) of a patient. In some aspects, the methods disclosed herein include informing the subject of a result of the P/F ratio (or another lung function parameter) determination assay and/or of a diagnosis based at least in part on P/F ratio (or another lung function parameter). The patient can be informed verbally, in writing, and/or electronically. This diagnosis can also be recorded in a patient medical record.

The term “medical record” or “patient medical record” refers to an account of a patient's examination and/or treatment that typically includes one or more of the following: the patient's medical history and complaints, the physician's physical findings, the results of diagnostic tests and procedures, and patient medications and therapeutic procedures. A medical record is typically made by one or more physicians and/or physicians' assistants and it is a written, transcribed or otherwise recorded record and/or history of various illnesses or injuries requiring medical care, and/or inoculations, and/or allergies, and/or treatments, and/or prognosis, and/or frequently health information about parents, siblings, and/or occupation. The record may be reviewed by a physician in diagnosing the condition.

The medical record can be in paper form and/or can be maintained in a computer-readable medium. The medical record can be maintained by a laboratory, physician's office, a hospital, a healthcare maintenance organization, an insurance company, and/or a personal medical record website. In some aspects, a diagnosis, based at least in part on the determined P/F ratio, is recorded on or in a medical alert article such as a card, a worn article, and/or a radiofrequency identification (RFID) tag. As used herein, the term “worn article” refers to any article that can be worn on a subject's body, including, but not limited to, a tag, bracelet, necklace, arm band, or head band.

As used herein, the term “diagnosis” means detecting a disease or determining the stage or degree of a disease. Usually, a diagnosis of a disease is based on the evaluation of one or more factors and/or symptoms that are indicative of the disease. That is, a diagnosis can be made based on the presence, absence or amount of a factor which is indicative of presence or absence of the disease or disorder. Each factor or symptom that is considered to be indicative for the diagnosis of a particular disease does not need be exclusively related to the particular disease, e.g. there may be differential diagnoses that can be inferred from a diagnostic factor or symptom. Likewise, there may be instances where a factor or symptom that is indicative of a particular disease is present in an individual that does not have the particular disease.

The term “diagnosis” also encompasses determining the therapeutic effect of a drug therapy, e.g., AP therapy, or predicting the pattern of response to a drug therapy. The diagnostic methods may be used independently, or in combination with other diagnosing and/or staging methods known in the medical arts for a particular disease.

As used herein, the term “differential diagnosis” refers to the determination of which of two or more diseases with similar symptoms is likely responsible for a subject's symptom(s), based on an analysis of the clinical data. The term is also used to refer to the determination of whether a patient is susceptible to treatment with an AP depending on whether the determined P/F ratio (or another lung function parameter) in a patient is above or below a predetermined threshold level, or elevated or decreased relative to the level in one or more controls.

The term “prognosis” as used herein refers to a prediction of the probable course and outcome of a clinical condition or disease, e.g., sepsis or ARDS. A prognosis is usually made by evaluating factors or symptoms of a disease that are indicative of a favorable or unfavorable course or outcome of the disease. The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

The terms “favorable prognosis” and “positive prognosis,” or “unfavorable prognosis” and “negative prognosis” as used herein are relative terms for the prediction of the probable course and/or likely outcome of a condition or a disease, e.g., sepsis or ARDS. A favorable or positive prognosis predicts a better outcome for a condition than an unfavorable or negative prognosis. In a general sense, a “favorable prognosis” is an outcome that is relatively better than many other possible prognoses that could be associated with a particular condition, whereas an unfavorable prognosis predicts an outcome that is relatively worse than many other possible prognoses that could be associated with a particular condition. Typical examples of a favorable or positive prognosis include increased lung function, preservation of lung function, increase in P/F ratio (or another lung function parameter), and the like.

The disclosure includes methods of treating ARDS in a subject, or AP for use in a method of treating ARDS in a subject, based on the changes in expression of P/F ratio (or another lung function parameter). The disclosure provides a method of treating a patient having ARDS, or AP for use in a method of treating ARDS in a patient, wherein the method comprises: administering an AP to the patient if the patient is determined to have a lower or decreased P/F ratio (or another lung function parameter) in the patient compared to predetermined P/F ratio (or another lung function parameter) threshold levels, or compared to the P/F ratio (or another lung function parameter) in one or more controls.

The disclosure also provides a method of treating a patient having ARDS, or AP for use in a method of treating ARDS in a patient, wherein the method comprises: (a) measuring PO₂ and FiO₂ (or deriving pO₂ and FiO₂ from another measurable lung function parameter) and determining P/F ratio (or another lung function parameter), and (b) administering an AP to the patient if the patient has a lower or decreased P/F ratio (or another lung function parameter) compared to a predetermined P/F ratio (or another lung function parameter) threshold level, or compared to the level of P/F ratio (or another lung function parameter) in one or more controls.

Also provided is method of treating a patient having ARDS, or AP for use in a method of treating ARDS in a patient, wherein the method comprises: (a) determining P/F ratio (or another lung function parameter) in the patient, and (b) suspending or not initiating the administration of AP, e.g., RecAP, to the patient if the patient has a higher or increased P/F ratio (or another lung function parameter) compared to a predetermined P/F ratio (or another lung function parameter) threshold level, or compared to the level of P/F ratio (or another lung function parameter) in one or more controls.

The disclosure also provides a method of treating a patient having ARDS, or AP for use in a method of treating ARDS in a patient, wherein the method comprises: (a) measuring PO₂ and FiO₂ (or deriving pO₂ and FiO₂ from another measurable lung function parameter) and determining P/F ratio (or another lung function parameter) in the patient; and (b) determining whether P/F ratio (or another lung function parameter) is higher or increased, or lower or decreased compared to a predetermined P/F ratio (or another lung function parameter) threshold level. In some aspects the method further comprises administering or advising a healthcare provider to administer an AP, e.g., RecAP, to the patient if the patient is determined to have a lower or decreased P/F ratio (or another lung function parameter) compared to a predetermined P/F ratio (or another lung function parameter) threshold level, or compared to the P/F ratio (or another lung function parameter) level in one or more controls; or to suspend or deny the administration of an AP if the patient is determined to have a higher or increased P/F ratio (or another lung function parameter) compared to a predetermined P/F ratio (or another lung function parameter) threshold level, or compared to the P/F ratio (or another lung function parameter) level in one or more controls.

Also provided is a method of treating a patient having ARDS, or AP for use in a method of treating ARDS in a patient, wherein the method comprises: (a) measuring pO₂ and FiO₂ (or deriving pO₂ and FiO₂ from another measurable lung function parameter) and determining P/F ratio (or another lung function parameter) in the patient, and (b) administering an AP to the patient if the patient is determined to have a lower or decreased P/F ratio (or another lung function parameter) compared to a predetermined P/F ratio (or another lung function parameter) threshold level, or compared to the P/F ratio (or another lung function parameter) level in one or more controls; or suspending, not initiating, or denying the administration of an AP to the patient if the patient is determined to have a higher or increased P/F ratio (or another lung function parameter) compared to a predetermined P/F ratio (or another lung function parameter) threshold level, or compared to the P/F ratio (or another lung function parameter) level in one or more controls.

The disclosure also provides a method of measuring the efficacy or pharmacodynamics of an AP in a patient diagnosed with ARDS, comprising: (a) conducting a first determination of the patient's P/F ratio (or another lung function parameter); (b) administering the AP, e.g., RecAP; and (c) conducting a second determination of P/F ratio (or another lung function parameter) in the patient, wherein an increase of the P/F ratio (or another lung function parameter) in the second determination compared to the patient's P/F ratio (or another lung function parameter) in the first determination, indicates that the patient is responding to treatment with the AP, e.g., RecAP.

The disclosure also provides a method of measuring the efficacy or pharmacodynamics of an AP in a patient diagnosed with ARDS, comprising: (a) conducting a first determination of the patient's P/F ratio (or another lung function parameter); (b) administering the AP, e.g., RecAP; and (c) conducting a second determination of P/F ratio (or another lung function parameter) in the patient, wherein a decrease of the P/F ratio (or another lung function parameter) in the second determination compared to the patient's P/F ratio (or another lung function parameter) in the first determination, indicates that the patient is not responding to treatment with the AP, e.g., RecAP.

In some aspects, the second determination is conducted 1, 2, 3, 4, 5, 6, 7, 8, 9, 10, 11, 12, 13, 14, 15, 16, 17, 18, 19, 20, 21, 22, 23, 24, 25, 26, 27, or 28 days, or at 1, 2, 3, 4, 5, 6, 7, 8, 9, 10, 11, 12, 13, 14, 15 or 16 weeks, or at intervening times, after administering the AP, e.g., RecAP.

In certain aspects, in all the treatment methods disclosed herein, a “loading” dose of an AP is administered to achieve a desired level of lung function in the patient. If the AP loading dose does not affect the patient's lung function significantly a decision could be made to discontinue treatment—e.g., to switch to an alternative therapy.

If the loading dose results in increased lung function in the patient a decision could be made to reduce the AP dose size or frequency to a “maintenance” dose. It is important to note that the methods provided here are guidelines for a healthcare provider to administer treatment, and the ultimate treatment decision will be based on the healthcare provider's sound judgment.

The formulation, dosage regimen, and route of administration of an AP, e.g., RecAP, can be adjusted to provide an effective amount for an optimum therapeutic response according to the methods disclosed herein. With regard to the administration of an AP, the AP may be administered through any suitable means, compositions and routes known in the art. With regard to dosage regiments, a single bolus can be administered, several divided doses can be administered over time or the dose can be proportionally reduced or increased as indicated by the exigencies of the therapeutic situation.

### III. Alkaline Phosphatases (AP)

Alkaline phosphatase (AP; EC 3.1.3.1 according to IUBMB Enzyme Nomenclature), is an enzyme that catalyzes the reaction of a phosphatase monoester and H2O to an alcohol and phosphate. Other name(s) for AP are alkaline phosphomonoesterase; phosphomonoesterase; glycerophosphatase; alkaline phosphohydrolase; alkaline phenyl phosphatase; orthophosphoric-monoester phosphohydrolase (alkaline optimum). The systemic name of AP is phosphate-monoester phosphohydrolase (alkaline optimum).

AP is a wide specificity enzyme, it also catalyzes transphosphorylations. In humans and other mammals at least four distinct but related AP are known. They are intestinal, placental, placental-like, and liver/bone/kidney (or tissue non-specific) AP. The first three are located together on chromosome 2 while the tissue non-specific form is located on chromosome 1.

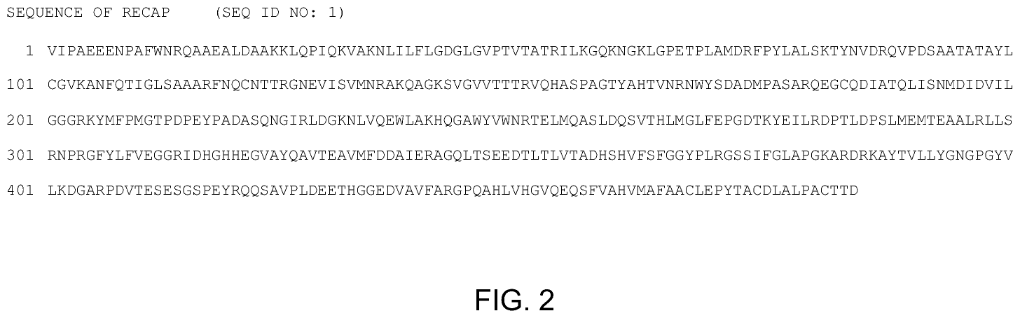

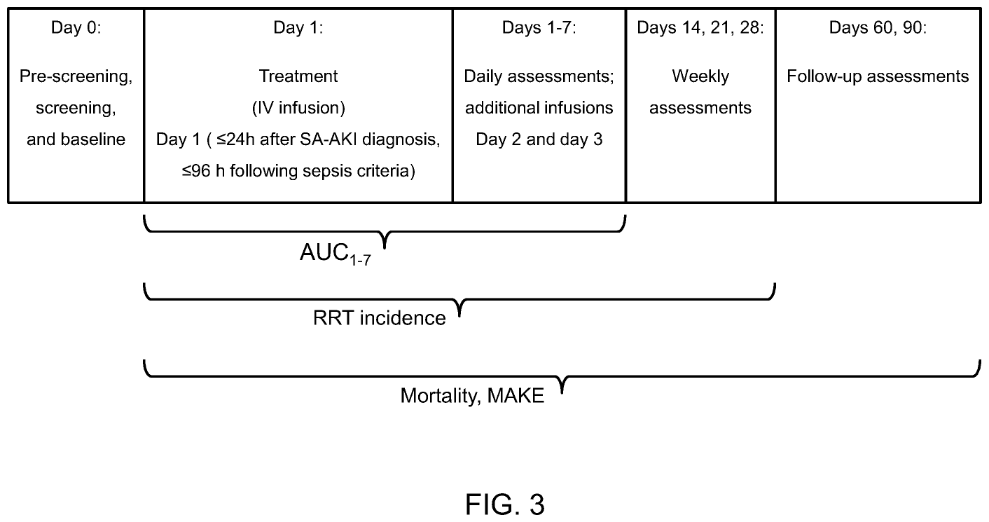

The term “AP of the present disclosure” refers to isolated alkaline phosphatases, including splice variants, isoforms, and polymorphic forms thereof. Also included are recombinant AP and chimeric AP. In a specific aspect, the AP is RecAP. The amino acid sequence of RecAP is shown in FIG. 2. In some aspects, an AP as used herein has at least about 70%, at least about 75%, at least about 80%, at least about 85%, at least about 90%, at least about 95%, at least about 96%, at least about 97%, at least about 98%, at least about 99%, or 100% sequence identity to the amino acid sequence of SEQ ID NO: 1. In some aspects, the AP is a functional fragment (i.e., a fragment of the AP, e.g., AP conserving at least about 10%, at least about 20%, at least about 30%, at least about 40%, at least about 50%, at least about 60%, at least 70%, at least about 80%, or at least about 90% of the AP activity of the corresponding full length AP). In some aspects, the AP is a variant or a derivative of an AP disclosed herein.

An AP for use according to the present disclosure can be a commercial AP enzyme, or any composition comprising the AP enzyme and any means capable of producing a functional AP enzyme in the context of the current invention, such as DNA or RNA nucleic acids encoding an AP protein.

The nucleic acid encoding AP may be embedded in suitable vectors such as plasmids, phagemids, phages, (retro)viruses, transposons, gene therapy vectors and other vectors capable of inducing or conferring production of AP. Also native or recombinant micro-organisms, such as bacteria, fungi, protozoa and yeast may be applied as a source of AP in the context of the current disclosure.

AP containing compositions for use according to the present disclosure can comprise a eukaryotic AP, e.g., a mammalian AP, which may be of the types tissue non-specific AP, such as liver-bone or kidney type, or tissue specific such as placental AP, intestinal AP and placental-like AP. The latter, also known as germ cell AP, is localized to testis, thymus and certain germ cell tumors, and is closely related to both the placental and intestinal forms of AP.

In some aspects, the mammalian AP is a human or a bovine AP. Non-limiting examples of a human AP sequence can be found in the NCBI (Genpept) collection and include: NP_001622 (intestinal AP), NP_001623 (placental AP), NP_112603 (placental-like AP) or NP_000469 (tissue non-specific AP). In some aspects, the AP comprises a polymorphism. In some aspects, the AP is placental AP, placental-like AP, intestinal AP, liver/bone/kidney AP, or a combination thereof. In some aspects, the AP is recombinant AP.

From a conformational point of view, an AP roughly consists of two domains: a crown domain and an active-site domain. The active-site domain can be divided in separate parts like the catalytic residue and the three metal ion sites (Zn1, Zn2 and Mg3). From a primary structure point of view, the crown domain is flanked by the amino acids that form the active site domain. The amino acid sequence of APs and the relative positions of the catalytic and crown domain are known by the skilled person.

In some aspects of the present disclosure, the AP is an isolated or recombinant AP comprising a crown domain and a catalytic domain, wherein said crown domain and said catalytic domain are obtained from different APs and wherein at least one of said different phosphatases is a human phosphatase. In some aspects, the AP is, for example, ECAP (Escherichia coli AP) or one of the seven known BIAPs (Bovine Intestinal AP).

In some aspects, AP is an isolated or recombinant AP comprising a crown domain and a catalytic domain, wherein said crown domain and said catalytic domain are obtained from different APs and wherein the different APs are human APs. This is especially useful if the modified phosphatase is subsequently used in human therapy. AP for use in the disclosed methods can be modified, e.g., genetically modified, APs of human origin which are not or only weakly immunogenic.

A modified AP disclosed herein can be used, for example, in “in vitro” or “ex vivo” diagnostics or treatments. Such modified phosphatase can comprise, for example, a human and an E. coli AP or may be composed of a bovine and an E. coli AP.

In some aspects of the present disclosure, the AP is an isolated or recombinant AP comprising a crown domain and a catalytic domain, wherein said crown domain and said catalytic domain are obtained from different APs and wherein said crown domain is the crown domain of placental AP (ALPP) and wherein said catalytic domain is the catalytic domain of intestinal AP (ALPI). In some aspects, at least one of the different APs is a human phosphatase. In other aspects, both different APs are human phosphatases.

Domain swapped mutants suitable for the methods disclosed herein which are based on the human APs are listed in TABLE 1.

TABLE 1
Domain swapped alkaline phosphatase enzymes. ALPI is intestinal AP,
ALPP is placental AP, GCAP is placental-like (or Germ Cell) AP and
TNAP is tissue non-specific AP.

| Catalytic domain | Crown domain | Referred to as |
|---|---|---|
| ALPI | GCAP | catALPI/crownGCAP |
| | TNAP | catALPI/crownTNAP |
| | ALPP | catALPI/crownALPP |
| ALPP | GCAP | catALPP/crownGCAP |
| | TNAP | catALPP/crownTNAP |
| | ALPI | catALPP/crownALPI |
| GCAP | ALPI | catGCAP/crownALPI |
| | ALPP | catGCAP/crownALPP |
| | TNAP | catGCAP/crownTNAP |
| TNAP | ALPI | catTNAP/crownALPI |
| | ALPP | catTNAP/crownALPP |
| | GCAP | catTNAP/crownGCAP |

In some aspects, the AP is a combination between the catalytic domain of ECAP or any of the human forms (ALPI, ALPP, GCAP or TNAP) with the crown domain of BIAP. Moreover, combinations of the catalytic domain of BIAP with the crown domain of any of the human forms can also be produced.

In some aspects, the modified AP is an AP which under natural conditions is linked to the membrane of a cell via a glycosylphosphatidylinositol (GPI) anchor but which is modified such that it is no longer attached to the membrane of a cell. All isoenzymes are functionally active in the cell membrane and GPI-anchor deficient forms are not naturally present at detectable levels. Although serum AP activity has been demonstrated it is generally accepted that the enzyme is still present in shed membrane fractions or membrane vesicles. AP activity in milk is also present in fractions containing membrane vesicles. The GPI anchor is stored as a precursor molecule in the cell where it is attached to the attachment site through a transamidase. The backbone of the GPI-anchor is identical in mammals, but cell-type dependent modifications are known.

In some aspects, for treatment of human subjects, the AP is human. This is preferred, due to the fact that AP forms obtained from other species may be immunogenic in human subjects and treatment could elicit immunological reactions and pathological side effects. In some subjects even lethal side effects, i.e., anaphylactic shock may occur and the risks of immunological side effects are therefore preferably minimized by use of human AP forms.

As isolation of AP from humans is not practical, human recombinant forms of AP proteins can be routinely produced in different recombinant expression platforms. However, expression and purification of GPI modified and membrane-anchored proteins is notoriously difficult; GPI proteins are difficult to separate from membranes and difficult to isolate and purify. Thus, in some aspects, the recombinant APs comprises a modification in the GPI signal sequence, wherein said modification results in a secreted AP, i.e., the AP is not attached to the cell membrane.

There is no general sequence responsible for the attachment of a GPI anchor, but there some specific consensus characteristics:
(i) hydrophobic stretch of amino acids at the C-terminus (at least 11 amino acids, but preferably more than 11 amino acids);
(ii) upstream of the hydrophobic region, a spacer of hydrophilic amino acids (5-12 amino acids);
(iii) GPI is attached to a small amino acid: glycine, aspartic acid, asparagine, alanine, serine or cysteine; and,
(iv) the 2 subsequent amino acids downstream of the GPI attachment site must be small amino acids and in the majority of cases they are selected from glycine, aspartic acid, asparagine, alanine, serine or cysteine.

In some aspects, the recombinant AP comprises a modification in the GPI signal sequence, wherein said modification results in a secreted AP that is biological active, i.e., it shows activity towards a biologically relevant substrate. In some aspects, the secreted AP is a human AP. In some aspects, the secreted human AP is human liver-kidney-bone phosphatase, human intestinal AP, or human placental-like alkaline phosphatase.

Based on the consensus characteristics above, a skilled person can introduce modifications, e.g., by inserting one or multiple amino acids that would disrupt part of the consensus, and would result in an AP not capable to attaching a GPI anchor. Thus, in some aspects, the recombinant AP comprises a modification in the GPI signal sequence which results in a secreted AP, wherein the modification comprises a mutation or a deletion of at least one amino acid in the sequence encompassing the consensus GPI signal sequence.

In some aspects, the AP is an AP disclosed in U.S. Pat. No. 8,557,545. In some aspects, the AP is a chimeric AP or chimeric AP-like protein such as those described in U.S. Patent Appl. Publ. No. US2017/0009216 and US2014/0193388. Preferably, the AP is a recombinant alkaline phosphatase comprising the catalytic domain of ALPI (intestinal alkaline phosphatase) and the crown domain of ALPP (placental alkaline phosphatase. More preferably, the AP is RecAP (corresponding to SEQ ID NO:1 and described in U.S. Patent Appl. Publ. No. US2017/0009216).

In some aspects, an AP of the present disclosure comprises (i) a sequence having at least about 70%, at least about 75%, at least about 80%, at least about 85%, at least about 90%, at least about 95%, at least about 96%, at least about 97%, at least about 98%, or at least 99% sequence identity with the crown domain of a human ALPP, and (ii) a sequence having at least about 70%, at least about 75%, at least about 80%, at least about 85%, at least about 90%, at least about 95%, at least about 96%, at least about 97%, at least about 98%, or at least 99% sequence identity with the catalytic domain of a human ALPI.

In some aspects, said sequence having said sequence identity to the crown domain of ALPP is situated in a protein according to the invention at approximately the same position as the crown domain of ALPP in the native ALPP protein

The percentage of identity of an amino acid or nucleic acid sequence, or the term “% sequence identity”, is defined herein as the percentage of residues in a candidate amino acid or nucleic acid sequence that is identical with the residues in a reference sequence after aligning the two sequences and introducing gaps, if necessary, to achieve the maximum percent identity. In a preferred embodiment, the calculation of said percentage of sequence identity is carried out without introducing gaps. Methods and computer programs for the alignment are well known in the art, for example “Align 2” or the BLAST service of the National Center for Biotechnology Information (NCBI).

In some embodiments, the invention provides an alkaline phosphatase (AP) for use in a method to treat acute respiratory distress syndrome (ARDS) in a subject in need thereof, comprising administering an effective amount of alkaline phosphatase (AP) to said subject, wherein the subject has moderate or severe ARDS prior to the treatment with AP, and the administration of the AP results in an increase in respiratory function. In a preferred embodiment, the subject has severe ARDS prior to the treatment with AP. In another preferred embodiment, the subject has moderate ARDS prior to the treatment with AP.

In some preferred embodiments, the AP is administered in at least one 500 U/kg to 2,000 U/kg dose. Preferably, the ARDS is associated with or due to sepsis, or the ARDS is associated with or due to a virus infection. In case the ARDS is associated with or due to sepsis, it is preferred that sepsis is detected less than 96 hours prior to AP administration, more preferably less than 72 hours prior to ARDS detection. It is preferred that treatment is initiated within 24 hours after sepsis is detected.

In some preferred embodiments, the AP is a human AP and/or the AP is a recombinant AP. If the AP is recombinant it is preferably a chimeric AP. Preferably, the chimeric AP has at least 80%, at least 85%, at least 90%, at least 95%, at least 96%, at least 97%, at least 98%, at least 99%, or 100% sequence identity to the amino acid sequence of RecAP (SEQ ID NO: 1).

In some preferred embodiments, an AP for use according to the invention is provided, wherein the increase in respiratory function comprises an increase in P/F ratio with respect to the P/F ratio in the absence of treatment with AP.

In some preferred embodiments, an AP for use according to the invention is provided, wherein treatment is initiated within 24 hours after ARDS is detected.

In some preferred embodiments, AP is administered once daily and/or administered intravenously. Preferably, AP is administered in three daily doses. Preferably, each AP dose is about 0.8 mg/kg or about 1.6 mg/kg of RecAP and/or the AP dose is about 500 U/kg or about 1000 U/kg of RecAP. With “about” in this context is meant +/−20%, preferably +/−10%, most preferably +/−5% of the indicated dose. For an average person (such as a person weighing between about 60-80 kg, each dose would be between about 30,000 U-about 160,000 U, preferably between about 30,000 U and about 80,000 U, more preferably between about 60,000 U and about 80,000 U. The dose in mg would amount to between about 48 mg and about 256 mg, preferably between 48 mg and about 128 mg, more preferably between 96-128 mg. Further provided is therefore an AP for use according to the invention, wherein the dose is between about 30,000 U and about 160,000 U, preferably between about 30,000 U and about 80,000 U, more preferably between about 60,000 U and about 80,000 U. Also provided is an AP for use according to the invention, wherein the dose is between about 48 mg and about 256 mg, preferably between 48 mg and about 128 mg, more preferably between 96-128 mg.

In some preferred embodiments, an AP for use according to the invention is provided, wherein the administration of at least one dose of AP results in a shortening of duration or cessation of mechanical ventilation therapy in a subject undergoing mechanical ventilation therapy.

Preferably, the administration of at least one dose of AP results in the preservation or increase of the P/F ratio in the subject. More specifically, AP for use according to the invention results in a decrease in mortality risk of the subject. Treatment of one of more subjects that are part of a group of subjects suffering from moderate or severe acute respiratory distress syndrome will result in a reduction of mortality rate of said group of subjects.

While the current application may describe features as part of the same embodiment or as parts of separate embodiments, the scope of the present invention also includes embodiments comprising any combination of all or some of the features described herein.

The invention is further explained in the following examples. These examples do not limit the scope of the invention, but merely serve to clarify the invention.

### EXAMPLES

### Example 1

### Human Recombinant Alkaline Phosphatase for Acute Respiratory Distress Syndrome

Acute respiratory distress syndrome (ARDS) adversely affects long-term lung outcomes and survival. Administration of the detoxifying enzyme alkaline phosphatase improved lung inflammation parameters in preclinical studies. We investigated the efficacy and safety of a human recombinant alkaline phosphatase (RecAP) (FIG. 2) in patients with ARDS.

In Part 1 of the adaptive phase 2a/2b STOP-AKI trial, patients were randomized to receive RecAP 0.4, 0.8, or 1.6 mg/kg, or placebo, once daily for 3 days to establish the optimal dose.

In Part 2, this dose was compared with placebo. The primary endpoint was time-corrected area under the curve of the endogenous creatinine clearance for days 1-7 (AUC₇ ECC), with renal replacement therapy (RRT) as a key secondary endpoint. Lung function was also analyzed. Overall, 301 patients were enrolled. No differences in (serious) adverse events between RecAP and placebo were observed between groups. RecAP appeared safe and well tolerated.

### Methods

STOP-AKI Trial Design and Participants

The STOP-AKI trial, was an international randomized, double-blind, placebo-controlled, four arm, parallel-group, dose-finding adaptive phase 2a/2b trial conducted in critically ill adults with sepsis-associated acute kidney injury. The protocol and main results of the STOP-AKI study were published previously (Pickkers P, Mehta R L, Murray P T, et al. Effect of Human Recombinant Alkaline Phosphatase on 7-Day Creatinine Clearance in Patients With Sepsis-Associated Acute Kidney Injury: A Randomized Clinical Trial. JAMA. 2018; 320(19):1998-2009).

ICU patients aged ≥18 years with a diagnosis of sepsis (Levy et al. Crit Care Med 2003; 31:1250-6) and a first diagnosis of AKI (Mehta et al. Crit Care 2007; 11:R31) were eligible for study participation. The inclusion and exclusion criteria and main results are described in Pickkers et al (JAMA 2018).

Outcome Measures

The primary objective of this study was to investigate the optimal therapeutic dose of RecAP and to evaluate its effect on kidney function. Effects on other organs, such as lung, were also investigated.

Safety, tolerability, pharmacokinetics (in the first 120 patients), immunogenicity, systemic and urinary biomarkers, and the effect of RecAP on quality of life in patients with SA-AKI and other non-kidney parameters were also investigated. Lung function analyses are described in detail below.
Lung function as assessed by fraction partial pressure arterial oxygen (PaO₂)/fraction of inspired oxygen (FiO₂) (P/F ratio) Carrico index, positive end expiratory pressure (PEEP), and tidal volume in mechanically ventilated patients.
Mechanical ventilator-free days: Days alive and not dependent on mechanical ventilation from randomization to day 28, inclusive #. A ventilator-free day was defined as a day on which a patient was not on ventilator (invasive or non-invasive mechanical ventilation).
Ventilator days: Days from start of first administration of study drug to being off mechanical ventilator (from day 1 to day 28, inclusive) for those patients who were on mechanical ventilator at the start of this period.

Statistical Analysis

Data were analyzed according to the intention-to-treat principle for patients from whom informed consent was obtained and who were randomized to a treatment arm. Details related to the sample size calculations are described below.

Sample Size Calculations

A sample size was planned of n₁=30 patients per treatment group in Part 1 with an additional n₂=85 patients recruited to the optimal RecAP-dose and placebo treatment groups in Part 2 (for a total sample size of n=290 patients). Custom-programmed simulations were performed using SAS software V.9.2 to determine power and type I error rate of the chosen sample size and design in a number of different dose-response scenarios. Each scenario assumed a standard deviation of 49 ml/min for the primary end point with an assumed response of 60 ml/min for the placebo group, and between 60 mL/min (no treatment effect) and 79 ml/min (strong treatment effect) for the RecAP dose groups.

Fifty thousand simulations were performed to show that the one-sided type I error rate is 2.4% (and hence is well controlled at the one-sided 2.5% significance level). The power was defined as the probability of rejecting the null hypothesis (of no difference between treatment groups) when one or more RecAP dose groups have a treatment effect, defined as a response of 69.5 ml/min. This was investigated across seven scenarios with 10 000 simulations performed for each. The chosen design achieved power of between 79% and 86% for scenarios with strong treatment effects for the medium and high RecAP dose groups; a varying response for the low-dose group, and between 66% and 67% when only the high-dose group had a strong treatment effect. As the sample size determination was based on the number of patients required for the intention-to-treat (ITT) analysis, patients who are randomly assigned and subsequently withdrew prior to completion of the study were not to be replaced.

A per-protocol analysis compared the intervention groups with the placebo group for patients who received study medication according to the study protocol, and had no more than two missing ECC values on days 1 through 7, as detailed in the statistical analysis plan.

For the descriptive statistics, continuous variables are presented as mean with standard deviation or median with interquartile range, depending on their distribution. Normally distributed variables were compared using Student's t test; Mann-Whitney U tests were used to compare non-normally distributed variables. Categorical (and binary) variables are presented as numbers with percentages and analyzed using chi-squared tests. Survival analyses with Kaplan-Meier curves were used for graphical presentation. Cox proportional-hazard regression analyses were used to estimate the hazard ratio for survival and for the number of RRT-, shock-, and mechanical ventilation-free days during study days 1-28 with the use of RecAP versus placebo.

The analysis of the primary efficacy endpoint was performed by an analysis of variance. All analyses performed on the secondary endpoints were for exploratory purposes only, therefore no multiplicity adjustment was required. A post-hoc multivariate analysis was conducted to establish the robustness of the RecAP effect. All statistical tests (SAS software version 9.4; SAS Institute Inc., Cary, NC, U.S.A.), performed on the intention-to-treat population, were two-sided using a 5% significance level.

### Results

Participants

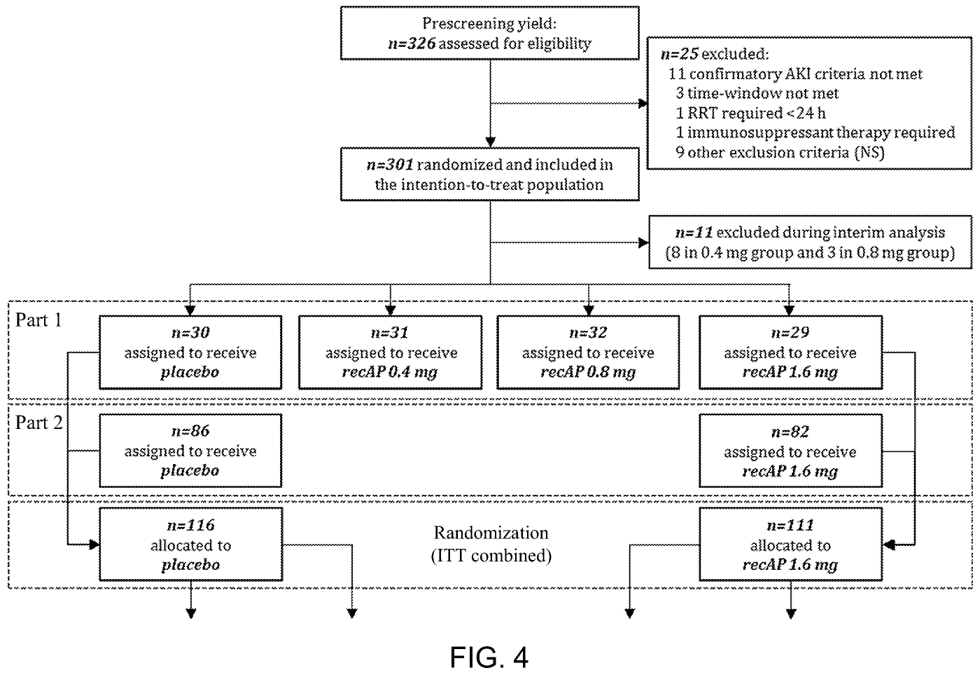

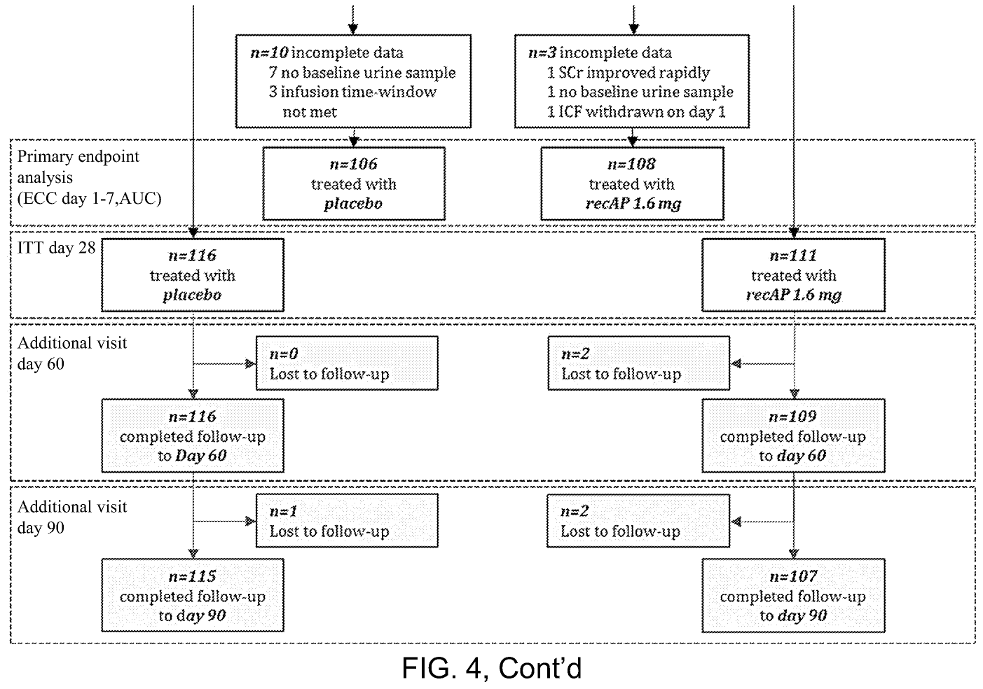

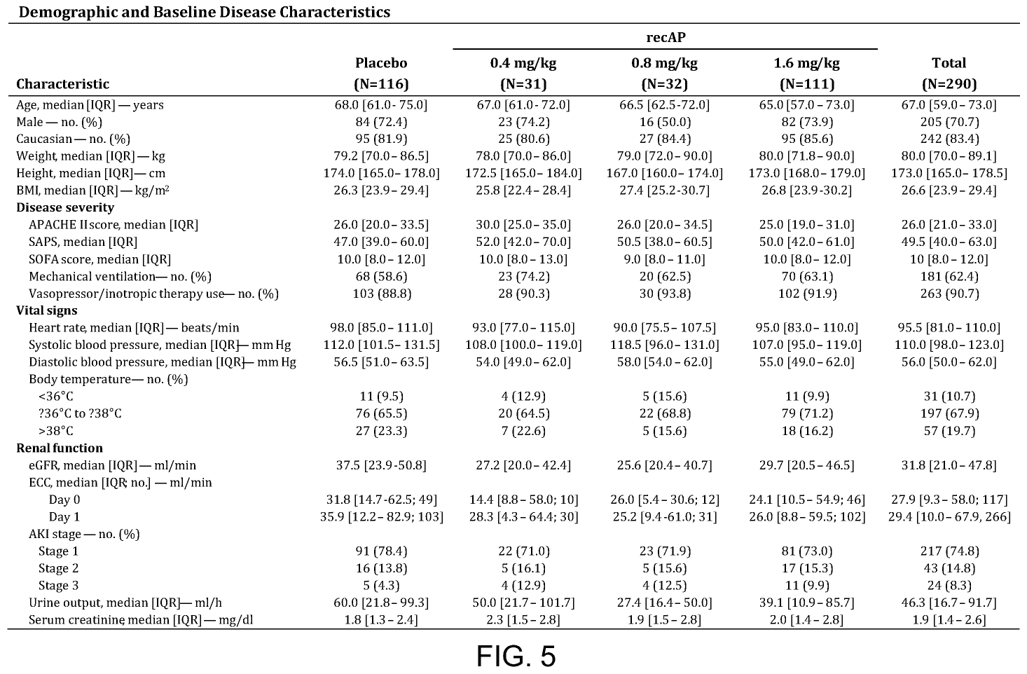

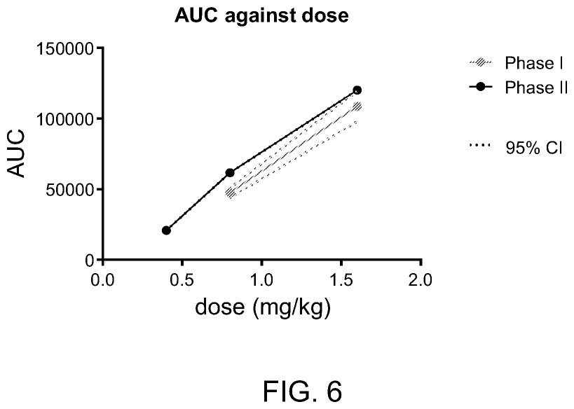

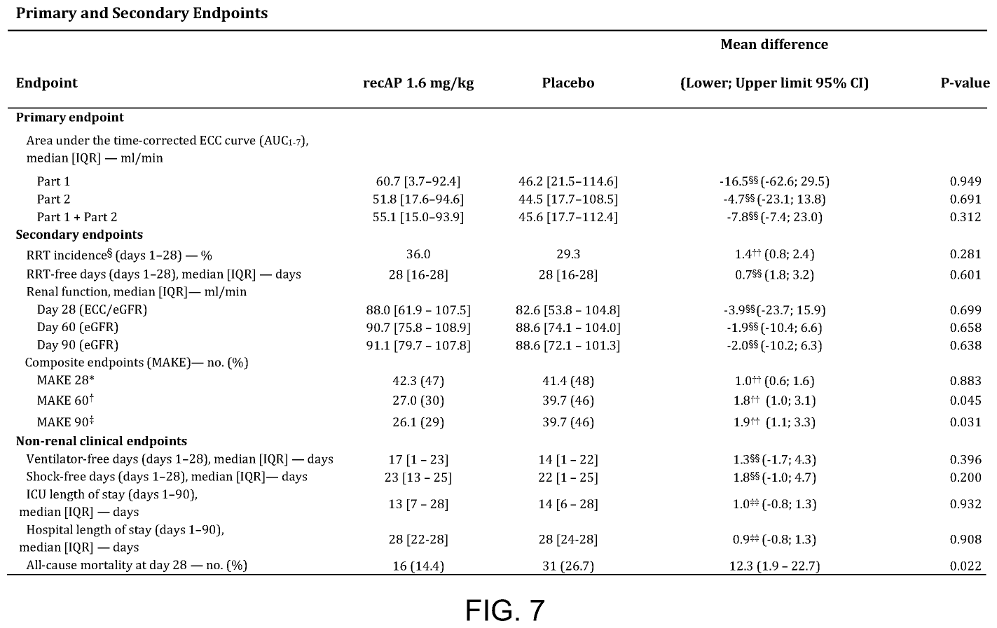

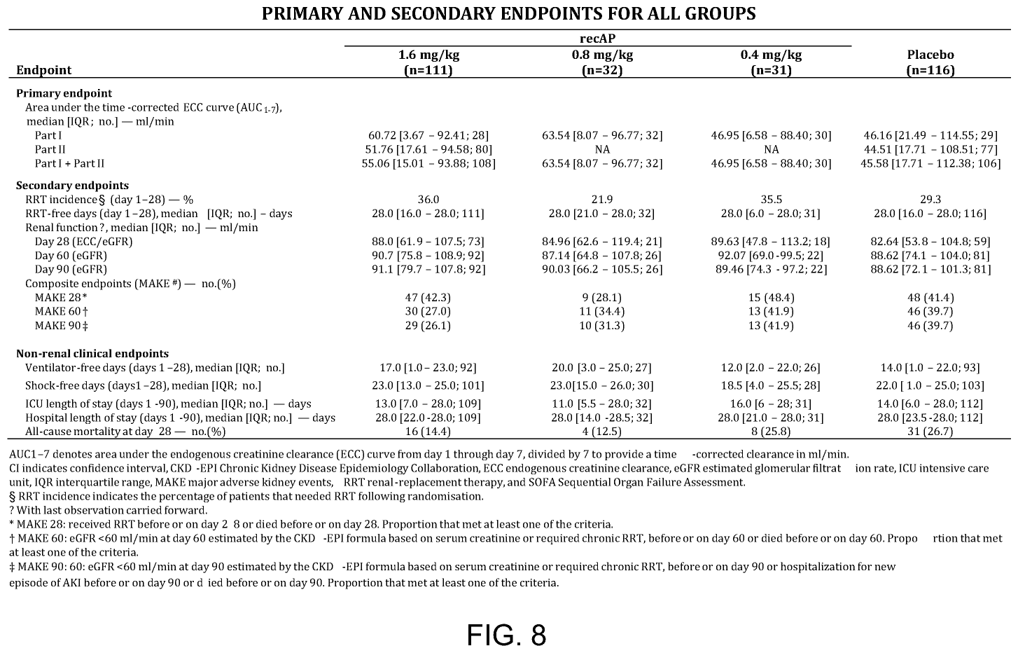

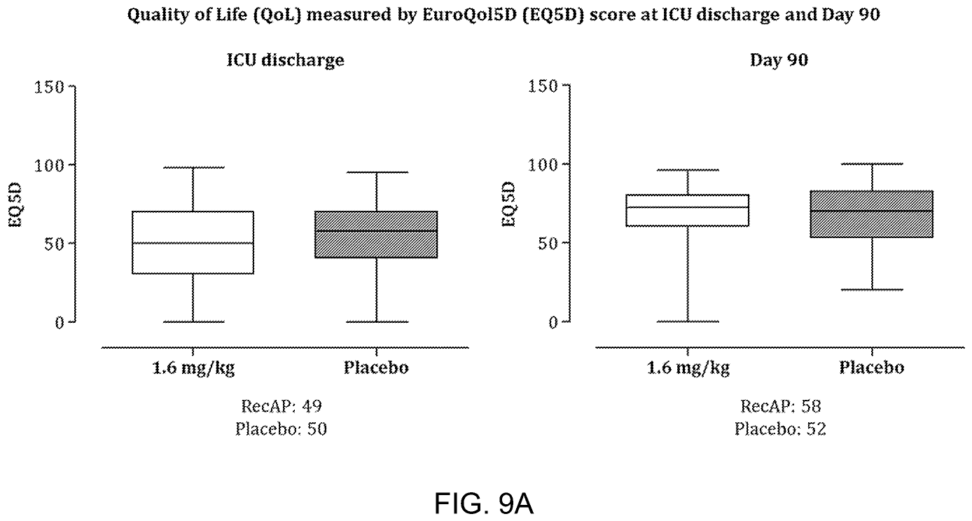

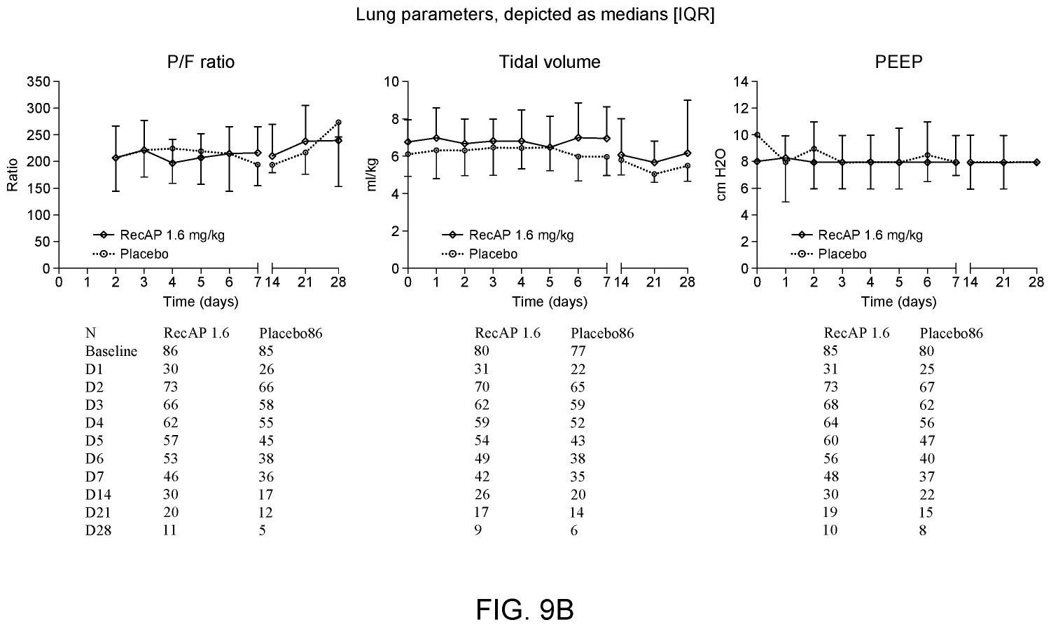

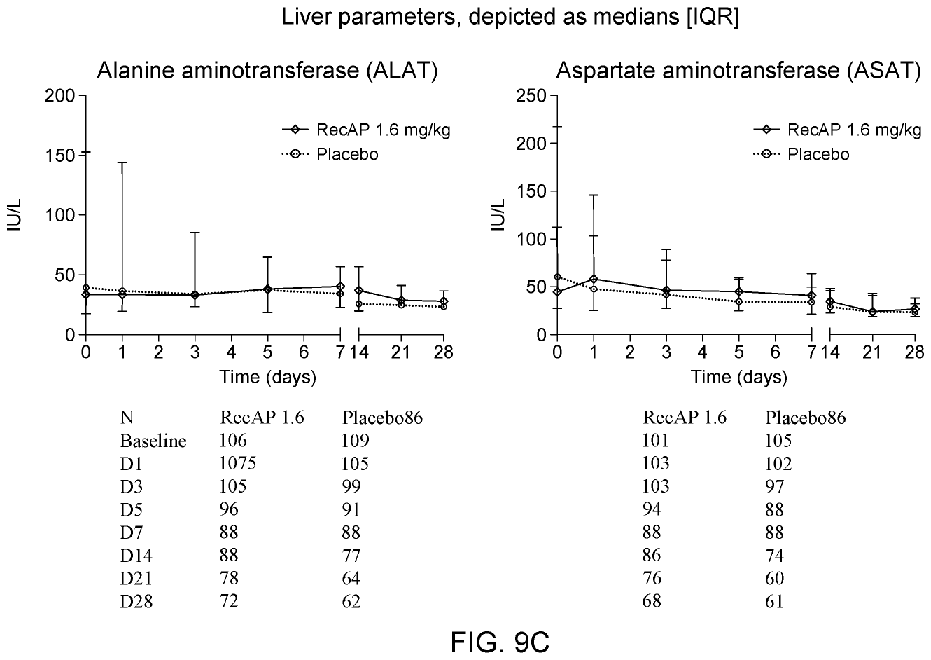

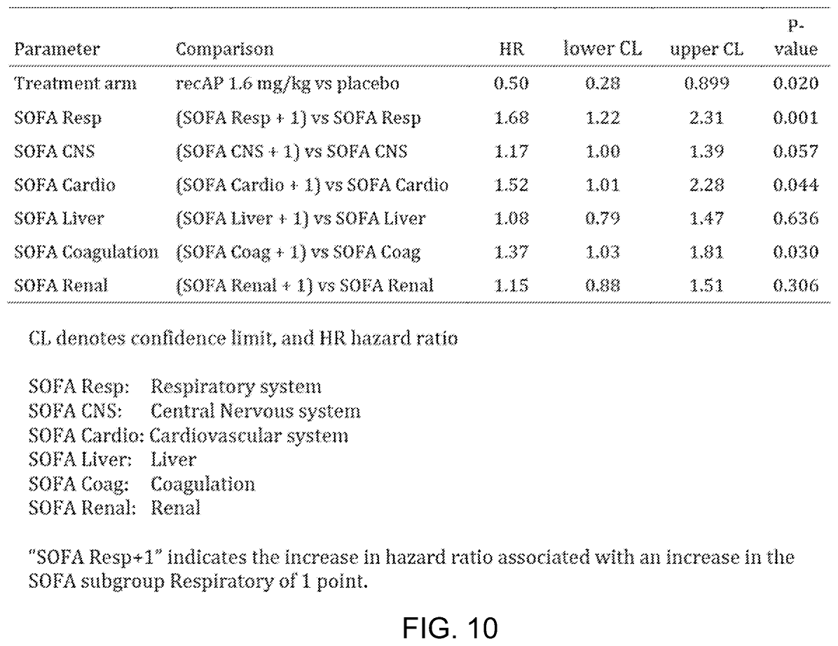

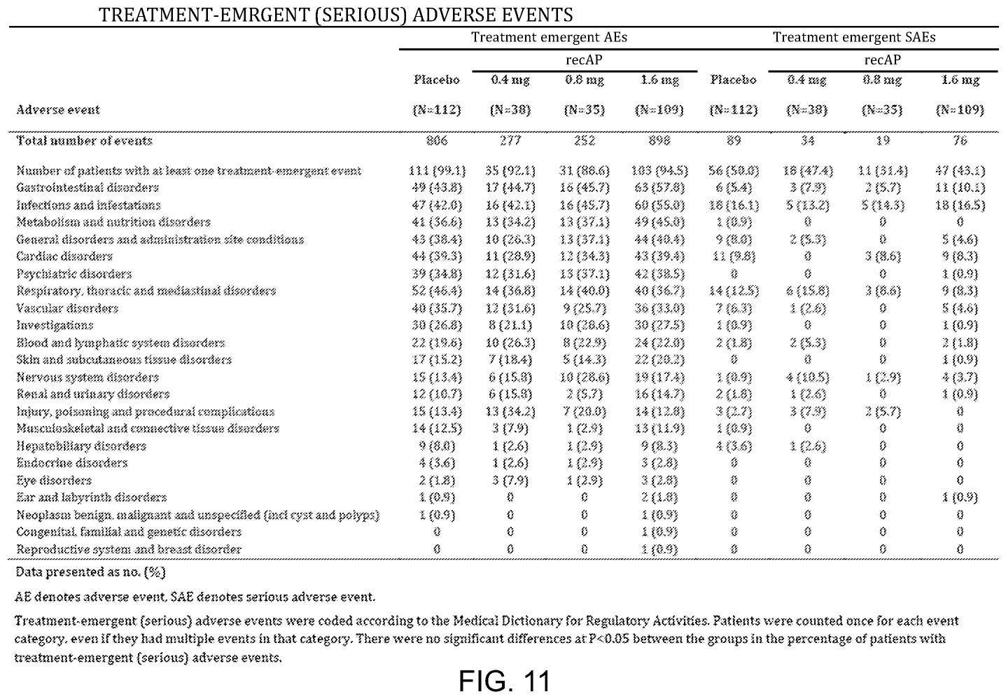

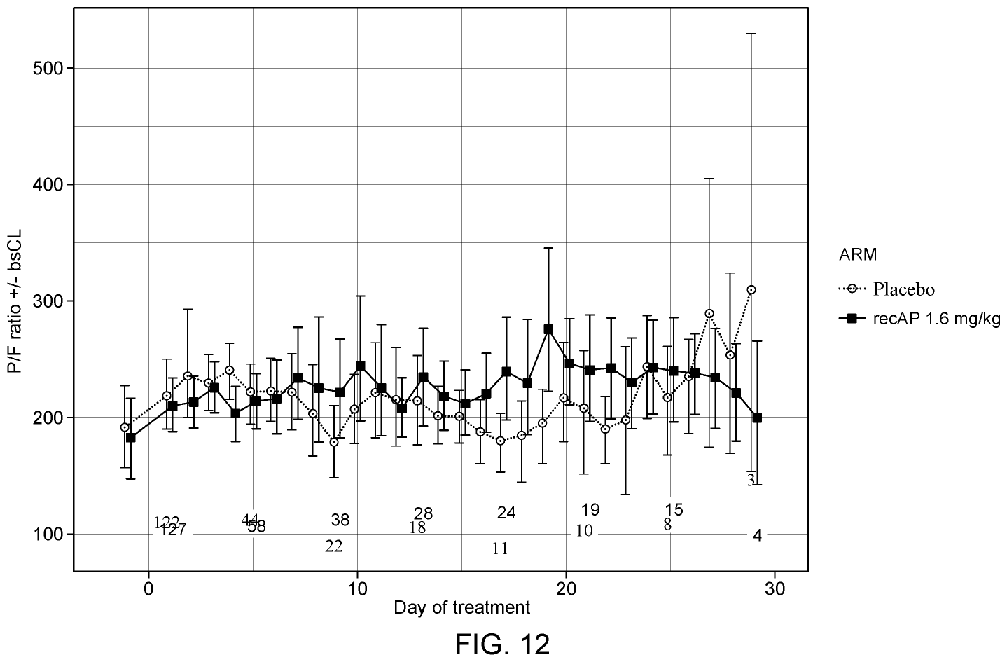

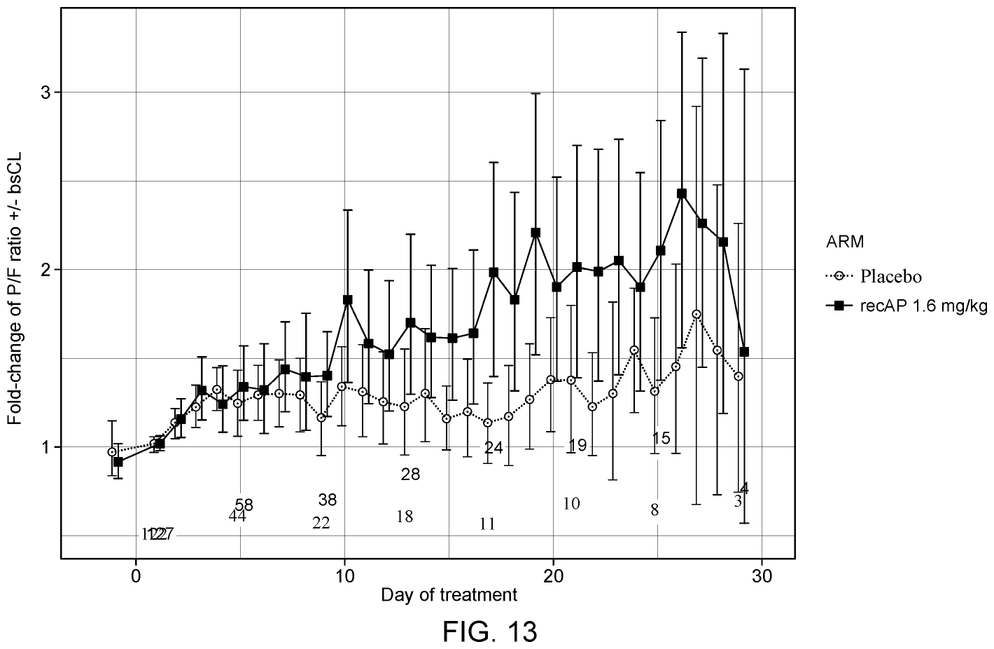

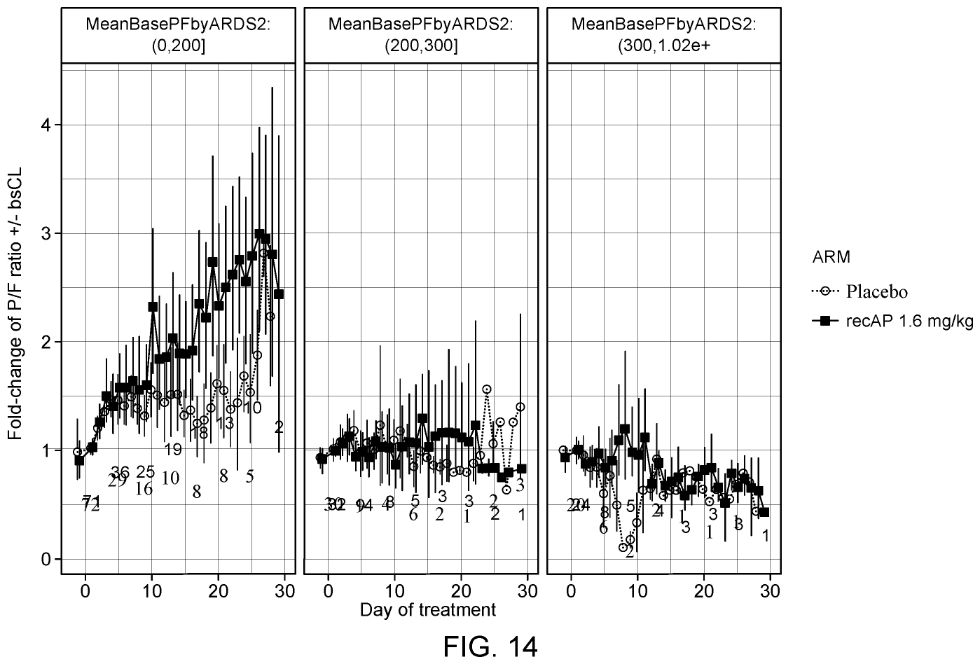

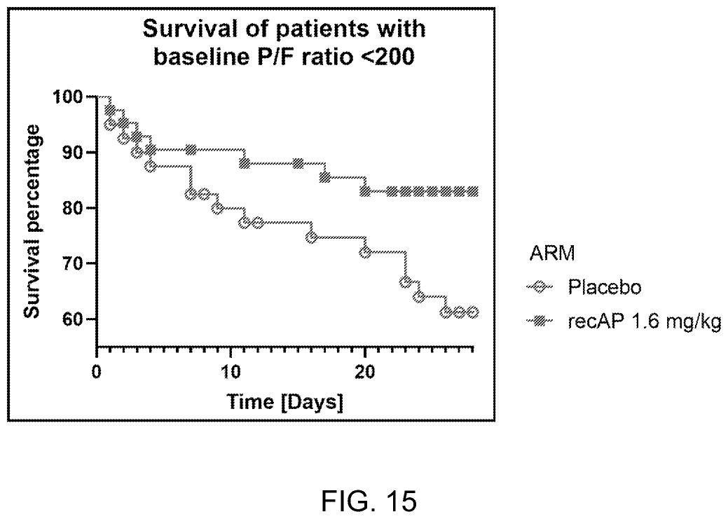

Of 326 patients who passed an initial screen, 301 patients were enrolled (FIG. 4) across 53 sites in 11 countries in the European Union and North America. Patients received RecAP 0.4 mg/kg, (n=30), 0.8 mg/kg, (n=32), 1.6 mg/kg (n=29 in Part 1 and n=82 in Part 2) or placebo (n=30 in Part 1 and n=86 in Part 2). Randomization resulted in well-balanced demographic and patient characteristics, except for slight baseline differences in renal function between groups (FIG. 5).

Safety

Treatment emergent Serious Adverse Events were reported in 43% of patients that received RecAP 1.6 mg/kg and in 50% of patients that received placebo. See FIG. 11. No RecAP dose-dependency in the incidence and nature of Adverse Events (AE's) was observed. Anti-drug antibody titers were just above the detection limit in 9 patients treated with RecAP.

Discussion

In this multinational double-blind, randomized controlled trial involving patients with SA-AKI, we observed that addition of RecAP to standard of care had a large impact on survival, associated with an improved and sustained recovery of renal function and longer-term clinical outcomes, including composite MAKE endpoints. Because this was a dose-finding proof-of-principle phase 2 trial, it was designed to include endpoints related to both renal dysfunction (e.g. short-term ECC) and the long-term, more patient-centered, clinical outcomes (e.g. MAKE_{day60-90}, survival).

In conclusion, RecAP therapy was considered safe and well-tolerated. In sepsis patients with AKI, RecAP treatment was observed to provide significant improvements of longer-term renal function, MAKE₆₀₋₉₀, and survival.

### Example 2

### Determination of RecAP Enzyme Activity and Protein Concentration

Activity Assay:

Determination of recAP enzyme activity was based on the conversion (hydrolysis) of 4-nitrophenolphosphate into 4-nitrophenol which is colored yellow. The change in optical density at 405 nm per unit time is a measure for the alkaline phosphatase activity. The assay buffer consisted of 0.25M Glycine buffer pH 9.6 at 25° C. with 2 mM MgCl₂ and 0.1 mM ZnCl₂ and 8.5 mM 4-nitrophenolphosphate.

The unit (U) definition for recAP, expressed as U/mL, is the amount of enzyme causing the hydrolysis of 1 pmol of 4-nitrophenolphosphate per minute at pH 9.6 and 25° C.

Protein Concentration:

Determination of total protein concentration in RecAP Drug Substance and Drug Product was performed by UV/Vis analysis. A RecAP solution is analysed at 280 nm and the absorbance is a measure for protein content (mg/mL) using the formula:

Concentration (mg/mL)=[A/(a×b)]×DF

with A=A280; b=path length; a=mass extinction coefficient of 1.01 mL mg−1 cm−1; DF is dilution factor.

### Example 3

### Exploration P/F Ratio

In preparation of a study into potential beneficial effects in the treatment of COVID-19, it is of interest to explore the effects of recAP (recombinant alkaline phosphatase) on relevant biomarkers or endpoints as observed in the STOP-AKI study (described in Example 1 and in Pickkers et al (JAMA 2018). One of the relevant endpoints is the ratio of partial pressure arterial oxygen and fraction of inspired oxygen, the P/F ratio. The objective of the current investigation is therefore: to explore the potential impact of recAP on P/F ratio as observed in the STOP-AKI study.

Relevant tables of the Analysis Data Model (ADaM) dataset as provided by AM-Pharma, specifically the Adverse Drug Reaction (ADRe) dataset, were read in into the statistical analysis program R (r-project.org; version 3.4.4 (2018 Mar. 15). The population of patients randomized to treatment that received either placebo or 1.6 mg/kg recAP, with P/F ratio measurements were included. For all patients that were measured with a P/F ration of 300 or lower, the PEEP was at least 5 cm H₂0, as prescribed in the Berlin 2012 definition. The baseline of P/F ratio values as determined in in screening samples and samples taken at Day 1 were averaged to increase stability of values and to reduce the occurrence of missing values, and labeled as MeanBase; patients with missing MeanBase values were excluded from the analysis. The absolute and normalized values are plotted as a function of time after the initial dose. The graphical exploration was also repeated after subsetting into tertiles of the MeanBase value. The average values were plotted together with bootstrapped confidence intervals. Trends observed in these plots were checked using linear regression and at relevant timepoints using the non-parametric test.

The progression of the ratio of partial pressure arterial oxygen and fraction of inspired oxygen, P/F ratio, over time after start of treatment is provided in FIGS. 12 to 14. The absolute P/F ratio does not change in a particular way over time when the complete dataset is taking into account, and there is no obvious difference between placebo and treated, see FIG. 12. The number of patients decreases with time into the study because of death or study discontinuation for other reasons and the width of bootstrapped confidence intervals increases accordingly. However, if the P/F ratio for each patient is determined as “fold-change” over the baseline value, recAP administered patients show an increase over time compared to placebo treated patients (FIG. 13).

As can be seen in FIG. 14, this difference between recAP treated and placebo treated patients is largely attributable to those patients that present with a P/F ratio lower than 200 mmHg at baseline (i.e. patients with moderate to severe ARDS), whereas only minor, transient, differences are seen in the mild ARDS group (200 mmHg <P/F ratio <300 mmHg) and in the group of patients that did not show ARDS at baseline (P/F ratio >300 mmHg). In conclusion, those patients that present with moderate to severe ARDS (P/F ratio <200 mmHg) at baseline benefit most from recAP treatment.

As can be seen in FIG. 15, in a group of patients with moderate to severe ARDS (P/F ratio <200 mmHG) at baseline, treatment with alkaline phosphatase resulted in a significant reduction in mortality when compared to placebo treated patients having a P/F ratio <200 mmHg. In one embodiment, therefore, AP for use according to the invention is provided, wherein the administration of at least one dose of AP results in a reduction in mortality risk in the subject. In a preferred embodiment, the reduction is a reduction in all-cause mortality risk. Preferably, the mortality risk in the subject is reduced relative to when said subject would not have been treated with AP. Also provided is AP for use according to the invention, wherein the administration of at least one dose of AP results in a reduction of mortality rate in a group of said subjects. In a preferred embodiment, the reduction is a reduction in all-cause mortality rate. Preferably, the mortality rate in the group of said subjects treated with AP is reduced relative to a group of subjects having severe to moderate ARDS that are not treated with AP.

Mortality rate, mortality risk, all-cause mortality rate and all-cause mortality risk have their ordinary meaning in the context of the present invention. Mortality rate or all-cause mortality rate in the present context means the death rate from all causes of death for a population in a given time period. Mortality risk or all-cause mortality risk in this context means the risk of death from all causes for an individual in a given time period.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. Alkaline phosphatase (AP) for use in a method for treating acute respiratory distress syndrome (ARDS) in a subject in need thereof, wherein the subject has moderate or severe ARDS.

2. AP for use according to claim 1, wherein the subject has moderate or severe ARDS with a P/F ratio of ≤200 mmHg when measured using a minimum positive end expiratory pressure (PEEP) of 5 cmH₂O.

3. AP for use according to claim 2, wherein the subject has severe ARDS with a P/F ratio of ≤100 mmHg prior to the treatment with AP.

4. AP for use according to claim 2, wherein the subject has moderate ARDS with a P/F ratio of >100 and ≤200 mmHg prior to the treatment with AP.

5. AP for use according to any one of claims 1 to 4, wherein the AP is administered in at least one 500 U/kg to 2,000 U/kg dose.

6. AP for use according to any one of claims 1 to 5, wherein the ARDS is associated with or due to sepsis.

7. AP for use according to any one of claims 1 to 6, wherein the ARDS is associated with or due to a virus infection.

8. AP for use according to any one of claims 1 to 7, wherein the AP is a human AP.

9. AP for use according to any one of claims 1 to 8, wherein the AP is a recombinant AP, preferably wherein the recombinant AP is chimeric.

10. AP for use according to any one of claims 1-9, wherein the AP has at least 80%, at least 85%, at least 90%, at least 95%, at least 96%, at least 97%, at least 98%, at least 99%, or 100% sequence identity to the amino acid sequence of RecAP (SEQ ID NO: 1).

11. AP for use according to any one of claims 1 to 10, wherein the administration of the AP results in an increase in respiratory function, preferably wherein the increase in respiratory function comprises an increase in P/F ratio of said subject, with respect to the P/F ratio of said subject in the absence of treatment with AP.

12. AP for use according to any one of claims 1 to 11, wherein said AP treatment is initiated within 48 hours after ARDS is detected.

13. AP for use according to any one of claims 1 to 12, wherein the administration of at least one dose of AP results in a shortening of duration or cessation of mechanical ventilation therapy in a subject undergoing mechanical ventilation therapy.

14. AP for use according to any one of claims 1 to 13, wherein AP is administered once daily.

15. AP for use according to any one of claims 1 to 14, wherein AP is administered intravenously.

16. AP for use according to any one of claims 1 to 15, wherein AP is administered in three daily doses.

17. AP for use according to any one of claims 1 to 16, wherein the AP dose is 0.8 mg/kg or 1.6 mg/kg of RecAP.

18. AP for use according to any one of claims 1 to 17, wherein the AP dose is 500 U/kg or 1000 U/kg of RecAP.

19. AP for use according to any one of claims 1 to 18, wherein the administration of at least one dose of AP results in the preservation or increase of P/F ratio in the subject.

20. AP for use according to any one of claims 1 to 19, wherein the administration of at least one dose of AP results in a decrease in mortality risk of the subject.

21. AP for use according to any one of claims 1 to 19, wherein the administration of at least one dose of AP results in a reduction of mortality rate of a group of subjects comprising said subject.


SEQUENCE LISTING (1 sequence)

SEQ ID NO: 1 - protein, 484 aa, Artificial Sequence
  RecAP
VIPAEEENPAFWNRQAAEALDAAKKLQPIQKVAKNLILFLGDGLGVPTVTATRILKGQKNGKLGPETPLAMDRFPYLALSKTYNVDRQVPDSAATATAYLCGVKANFQTIGLSAAARFNQCNTTRGNEVISVMNRAKQAGKSVGVVTTTRVQHASPAGTYAHTVNRNWYSDADMPASARQEGCQDIATQLISNMDIDVILGGGRKYMFPMGTPDPEYPADASQNGIRLDGKNLVQEWLAKHQGAWYVWNRTELMQASLDQSVTHLMGLFEPGDTKYEILRDPTLDPSLMEMTEAALRLLSRNPRGFYLFVEGGRIDHGHHEGVAYQAVTEAVMFDDAIERAGQLTSEEDTLTLVTADHSHVFSFGGYPLRGSSIFGLAPGKARDRKAYTVLLYGNGPGYVLKDGARPDVTESESGSPEYRQQSAVPLDEETHGGEDVAVFARGPQAHLVHGVQEQSFVAHVMAFAACLEPYTACDLALPACTTD